(19) United States
(12) Patent Application Publication
(10) Pub. No.: US 2021/0207075 A1
(43) Pub. Date: Jul. 8, 2021

(54) LYSIS COIL APPARATUS AND USES THEREOF FOR ISOLATION AND PURIFICATION OF POLYNUCLEOTIDES

(71) Applicant: VGXI INC., The Woodlands, TX (US)

(72) Inventors: Jared Nelson, Hockley, TX (US); Stephen Rodriguez, Houston, TX (US); Jeffrey E. Darnell, The Woodlands, TX (US)

(21) Appl. No.: 17/058,747

(22) PCT Filed: May 31, 2019
(86) PCT No.: PCT/US19/34840

§ 371 Date: Nov. 25, 2020

Related U.S. Application Data
(60) Provisional application No. 62/678,355, filed on May 31, 2018.

(51) Int. Cl. C12N 1/06; C12N 15/10

(52) U.S. Cl. CPC C12N 1/06; C12N 15/1003

(57) ABSTRACT

The invention provides a lysis coil apparatus that can be integrated into systems and processes for production of DNA plasmid.

### CROSS-REFERENCE TO RELATED APPLICATIONS

This application claims priority to U.S. Provisional Patent Application No. 62/678,355, filed May 31, 2018, the contents of which are incorporated by reference herein in its entirety.

### BACKGROUND OF THE INVENTION

Biomolecules are commonly processed and purified for various research and development purposes, and in many cases for the manufacture of biopharmaceuticals for treating patients. In particular, polynucleotides, including DNA plasmids, can be purified from host cells.

Increasing attention has been focused on the delivery of DNAs as therapeutic agents (i.e., DNA gene therapy) for the treatment of genetic diseases and for genetic immunization. Because of safety concerns with using potentially infectious viruses, researchers have been studying alternatives to viruses, using naked DNA or other non-viral methods of DNA delivery. As the demand for gene therapy increases, huge quantities of plasmids or appropriate DNA will be needed. However, limitations of current methods for isolating larger and larger amounts of DNA at purity levels necessary for human application may impede progress in this field.

Generally, methods of DNA plasmid manufacturing involve the steps of replicating the plasmid in host cells, lysing and releasing the plasmids from such cells, and then isolating the plasmids. This all needs to be performed while obtaining high purity levels necessary for clinical studies in humans, and at quantities necessary for providing appropriate dosage levels for clinical studies, and ultimately for commercial supplies.

There are a variety of existing methods to purify plasmids; however, these methods are not suitable for large scale preparations. Laboratory scale purification techniques cannot simply be scaled up for the volumes involved in large scale plasmid preparation. Large scale preparations require the optimization of yield and molecular integrity while maximizing removal of contaminants and concentration of plasmid. In producing large quantities of plasmid DNA at high concentration, a problem exists in maintaining the plasmid as supercoiled and open circle relaxed form. Storage conditions generally require high salt, and molecular degradation over time remains a problem even in the presence of salt. Many existing purification methods rely upon the use of potentially dangerous, toxic, mutagenic or contaminated substances, and/or expensive substances or equipment, which, again, are not desirable for large scale preparations. Some existing purification methods utilize enzymes to digest protein for eventual elimination and such enzymes are costly for large scale production and can cause a risk of biologic contamination.

There are a variety of ways to lyse host bacterial cells. Well-known methods used at laboratory scale for plasmid purification include enzymatic digestion (e.g. with lysozyme), heat treatment, pressure treatment, mechanical grinding, sonication, treatment with chaotropes (e.g. guanidinium isothiocyante), and treatment with organic solvents (e.g. phenol). Although these methods can be readily practiced at small scale, few have been successfully adapted for large-scale use in preparing plasmids. Currently, the preferred method for lysing bacteria for plasmid purification is through the use of alkali and detergent. This technique was originally described by Birnboim and Doly (1979, Nucleic Acids Res. 7, 1513 1523). A commonly used variation of this procedure is described on pp. 1.38 1.39 of Sambrook et al. (Molecular Cloning: A Laboratory Manual, 2.sup.nd Ed., Cold Spring Harbor Laboratory Press, Cold Spring Harbor, N.Y.). This lysis method has distinct advantages; in addition to providing efficient release of plasmid molecules from the cells, this procedure provides substantial purification of the plasmid by removing much of the host protein, lipids, and genomic DNA. Removal of genomic DNA is particularly valuable, since it can be difficult to separate it from plasmid DNA by other means. These advantages have made this a preferred method for lysing bacterial cells during plasmid purification at laboratory scale.

It has previously been believed that mixing a cell suspension and a lysis solution must be performed at very low shear forces. This has been described in regard to mixing suspensions of plasmid-containing bacteria with lysis solutions comprising alkali and detergent. U.S. Pat. No. 5,837,529 and U.S. Patent Publication No. 2002/0198372. Each contemplate using static mixers to achieve continuous low shear mixing, while U.S. Pat. No. 6,395,516 contemplates using a designed vessel for controlled mixing in batch mode. Such methods have clear drawbacks. In one regard, while striving to minimize excessive shear, mixing of the cell suspension with the lysis solution may be incomplete. In another regard, using static mixers limits process flexibility. As described in U.S. Patent Publication No. 2002/0198372, it is necessary to optimize the number of static mixing elements, as well as the flow rates of the fluids passing through the elements. Such optimization restricts the amount of material that may be processed in a given time with the optimized static mixing apparatus. This limits the ability to increase process scale, unless a new, higher-capacity static mixing apparatus is constructed and optimized. Use of batch mixing vessels, as described in U.S. Pat. No. 6,395,516, has comparable drawbacks. Achieving complete mixing in all regions of a batch mixing vessel is well known by those of skill in the art to be challenging. Furthermore, batch mixing vessels are poorly suited for applications that require a controlled exposure time wherein the cell suspension is contacted with the lysis solution. In particular, it is well known that prolonged exposure of plasmid-containing cells to alkali may lead to the formation of excessive amounts of permanently denatured plasmid, which is generally inactive, undesirable, and difficult to subsequently separate from biologically active plasmid. Typically, it is desirable to limit such exposure times to about 10 minutes or less. Achieving such limited exposure times is difficult or impossible using large scale batch mixing. Solutions to the above problems have in-part been described in US Patent Publication No. 2009/0004716 A1.

Thus, there still remains a need in the art for methods of large scale production of biologically active molecules of interest, such as plasmids, and in particular a need for a manufacturing apparatus including a lysis coil apparatus that is configured for large scale production, portable, disposable (one time use), and/or economical, and manufacturing methods using such apparatuses.

### SUMMARY OF THE INVENTION

An aspect of the present invention includes a lysis coil apparatus capable of fluidly receiving a solution of cell suspension and a lysis solution, and fluidly transferring said solutions as a solution mixture thereby lysing and releasing contents of cells in the cell suspension, comprising: a cylindrical lysis coil holder having a height, and a flexible lysis coil having a first end, a second end, and a length in-between, said flexible lysis coil configured to receive a solution of cell suspension and a lysis solution from the first end and transferring said mixture solution out of the lysis coil from the second end; wherein said lysis coil holder is capable of receiving and securing a flexible lysis coil onto outer surface of said cylindrical lysis coil holder. In some aspects of the invention, the lysis coil holder has a surface embedded with a uniform helical groove having a length traversing the height of the lysis coil holder, wherein said flexible lysis coil has an interior diameter of a size that enable the lysis coil to be received by the groove of the lysis coil holder and traverses the length of the groove of said lysis coil holder.

In some embodiments, the interior diameter of said lysis coil is less than about 1 inch, and can include ⅞, ¾, ⅝, ½, ⅜, or ¼ inch, and preferably the interior diameter of said lysis coil is ¾ in some embodiments, ½ inch in some embodiments, or ⅜ inch in other preferred embodiments. In some embodiments, the lysis coil is configured to flow the solution mixture at a linear flow rate resulting in retention time in the lysis coil between about 4 to about 6 minutes, and preferably about 5 minutes. While in some embodiments, the lysis coil is configured to allow the solution mixture to flow at a linear flow rate of between 8 m/min to about 12 m/min, and preferably at a linear flow rate of about 9.95, 9.90, 9.85, 9.80. 9.75, 9.70, 9.65, 9.60, 9.55, or 9.50 m/min, and more preferably 9.80, 9.75, or 9.70 m/min. The lysis coil can have a length greater than 100 feet long, and preferably 140, 145, 150, 155, 160, 165, or 170 feet long, and more preferably 150, 155 or 160 feet long. In some embodiments of the lysis coil apparatus, the lysis coil is disposable after a single use.

The lysis coil holder can have a radial diameter of about 20, 21, 22, 23, 24, 25, 26, 27, 28, 29, or 30 inches, and preferably 23, 24, or 25 inches, and a height between about 3 feet to about 6.25 feet, about 3.5 feet to about 6.25 feet, about 4 feet to about 6.25 feet, about 4.5 feet to about 6.25 feet, about 5.0 feet to about 6.25 feet, about 3.5 feet to about 6.0 feet, about 4 feet to about 6.0 feet, about 4.5 feet to about 6.0 feet, about 5.0 feet to about 6.0 feet, about 3.5 feet to about 5.75 feet, about 3.75 feet to about 5.75 feet, about 4 feet to about 5.75 feet, about 4.5 feet to about 5.75 feet, or about 5.0 feet to about 5.75 feet, and preferably about 5.8, 5.9, 6.0, 6.1, or 6.2 feet. In some embodiments of the lysis coil apparatus, the groove of the lysis coil holder traverses the circumference of the lysis coil holder at a pitch from about 2.15 degree to about 3.43 degree. The lysis coil holder can have wheel supports by which the lysis coil apparatus can be readily transported.

In some preferred embodiments of the lysis coil apparatus, the lysis coil has an interior diameter of about ⅜ inch, ½ inch, or ¾ inch, and the lysis coil is configured to allow the solution mixture to flow at a linear flow rate of about 9.70, 9.75, or 9.80 m/min. In some preferred embodiments of the lysis coil apparatus, the lysis coil has a length of about 150 feet, 155 feet, or 160 feet long and the retention time is about 4.8 min, 4.9 min, 5.0 min, 5.1 min, or 5.2 min. In other preferred embodiments, the linear flow rate is about 9.75 m/min and the length of the lysis coil is about 150 feet long. Further, in some preferred embodiments, the length is about 150 feet long and the lysis coil is configured to allow the solution mixture to flow at a linear flow rate of about 9.75 m/min. In other preferred embodiments, the lysis coil interior diameter is ⅜ inch and the lysis coil length is about 150 feet long.

Another aspect of the present invention is a method of lysing cells containing a desired polynucleotide using a lysis coil apparatus capable of fluidly receiving a solution of cell suspension and a lysis solution, and fluidly transferring said solutions as a solution mixture into contact with a neutralizing solution thereby lysing and releasing contents of cells in the cell suspension, comprising: a cylindrical lysis coil holder having a height, and a flexible lysis coil having a first end, a second end, and a length in-between, said flexible lysis coil configured to receive a solution of cell suspension and a lysis solution from the first end and transferring said mixture solution out of the lysis coil from the second end; wherein said lysis coil holder has a surface embedded with a uniform helical groove having a length traversing the height of the lysis coil holder with a consistent pitch; and wherein said flexible lysis coil has an interior diameter of a size that enables the lysis coil to be received by the groove of the lysis coil holder and traverses the length of the groove of said lysis coil holder, comprising the steps: securing a disposable lysis coil into the groove of the lysis coil holder; transferring the solution of cell suspension into the first end of the lysis coil; transferring the lysis solution into the first end of the lysis coil to enable the solution of the cell suspension to mix with the lysis solution; and fluidly transferring the solution mixture to a compartment along with a neutralizing solution to end the lysing process.

In some embodiments of the method of lysing cells, the transferring steps occur at a linear flow rate of from about 8 m/min to about 12 m/min. In other embodiments of the method of lysing cells, the mixture solution traverses the length of the lysis coil in about between 4 minutes to 6 minutes. In some embodiments, the transferring steps occur at a linear flow rate of about 9.75 m/min.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a side view of an exemplary lysis coil apparatus.

FIG. 2 depicts a top view of an exemplary lysis coil apparatus.

FIG. 3 depicts a magnified view of the top end of an exemplary lysis coil apparatus.

FIG. 4 depicts a cross-sectional view of a section of an exemplary lysis coil apparatus.

FIG. 5 depicts a technical diagram of an exemplary lysis coil apparatus.

FIG. 6A and FIG. 6B depict the results of measuring the relationship between coil hold time, fluid flow rate, and fluid linear velocity in lysis coils having: an inner diameter of ¾ inch and a length of 160 feet (FIG. 6A); and an inner diameter of ⅜ inch and a length of 150 feet (FIG. 6B).

FIG. 7A and FIG. 7B depict the results of purification data for Plasmid A.

FIG. 8 depicts the results of HPLC analysis of resuspended cells and different stages of lysate.

FIG. 9 depicts a summary of purification data for six plasmid lots.

FIG. 10A through FIG. 10C depict the results of plasmid purification tests using three different lysis coil apparatus configurations.

FIG. 11A and FIG. 11B depict the results of a review of the solutions used in five plasmid purification lots.

FIG. 12 depicts a table listing the results of six plasmid purification lots using two different lysis coil apparatus configurations.

FIG. 13 depicts a table listing the results of HPLC analysis of plasmid concentration from the six plasmid purification lots in FIG. 12.

FIG. 14 depicts a table listing bulk release testing results for the six plasmid purification lots in FIG. 12.

FIG. 15A and FIG. 15B depict a table listing the results of gel analysis of the lysis and Q process for the six plasmid purification lots in FIG. 12.

FIG. 16A through FIG. 16F depict the results of HPLC analysis of lysate samples.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

As used herein, the term “a” or “an” may refer to one or more than one. As used herein in the claims, when used in conjunction with the word “comprising”, the words “a” or “an” may mean one or more than one. As used herein, “another” may mean at least a second or more.

As used herein, the term “alkali” refers to a substance that provides a pH greater than about 8 when a sufficient quantity of the substance is added to water. The term alkali includes, but is not limited to, sodium hydroxide (NaOH), potassium hydroxide (KOH), or lithium hydroxide (LiOH).

As used herein, the term “detergent” refers to any amphipathic or surface-active agent, whether neutral, anionic, cationic, or zwitterionic. The term detergent includes, but is not limited to, sodium dodecyl sulfate (SDS), Triton (polyethylene glycol tert-octylphenyl ether, Dow Chemical Co., Midland, Mich.), Pluronic (ethylene oxide/propylene oxide block copolymer, BASF Corp., Mount Olive, N.J.), Brij (polyoxyethylene ether, ICI Americas, Bridgewater, N.J.), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Tween® (polyethylene glycol sorbitan, ICI Americas, Bridgewater, N.J.), bile acid salts, cetyltrimethylammonium, N-lauroylsarcosine, Zwittergent (n-alkyl-N,N-dimethyl-3-ammonio-1-propanesulfonate, Calbiochem, San Diego, Calif.), etc.

As used herein, the term “ion exchange” refers to a separation technique based primarily on ionic interactions between a molecule or molecules of interest, and a suitable ion exchange material. Although the ion exchange material may most commonly take the form of a chromatography resin or membrane, it may be any material suitable for performing separations based on ionic interactions. The term ion exchange encompasses anion exchange, cation exchange, and combinations of both anion and cation exchange.

As used herein, the term “anion exchange” refers to a separation technique based primarily on ionic interactions between one or more negative charges on a molecule or molecules of interest, and a suitable positively charged anion exchange material. Although the anion exchange material may most commonly take the form of a chromatography resin or membrane, it may be any material suitable for performing separations based on the described ionic interactions.

As used herein, the term “cation exchange” refers to a separation technique based primarily on ionic interactions between one or more positive charges on a molecule or molecules of interest, and a suitable negatively charged cation exchange material. Although the cation exchange material may most commonly take the form of a chromatography resin or membrane, it may be any material suitable for performing separations based on the described ionic interactions.

As used herein, the terms “hydrophobic interaction” and “HIC” refer to a separation technique based primarily on hydrophobic interactions between a molecule or molecules of interest, and a suitable primarily hydrophobic or hydrophilic material. Although the primarily hydrophobic or hydrophilic material may most commonly take the form of a chromatography resin or membrane, it may be any material suitable for performing separations based on hydrophobic interactions.

As used herein, the term “plasmid” refers to any distinct cell-derived nucleic acid entity that is not part of or a fragment of the host cell's primary genome. As used herein, the term “plasmid” may refer to either circular or linear molecules composed of either RNA or DNA. The term “plasmid” may refer to either single stranded or double stranded molecules, and includes nucleic acid entities such as viruses and phages.

As used herein, the term “genomic DNA” refers to DNA derived from the genome of a host cell. As used herein, the term includes DNA molecules comprising all or any part of the host cell primary genome, whether linear or circular, single stranded or double stranded.

As used herein, the term “endotoxin” refers to lipopolysaccharide material that is derived from Gram-negative bacteria and that causes adverse effects in animals. Endotoxin can typically be detected by the limulus amebocyte lysate (“LAL”) assay.

As used herein, the term “chromatography” includes any separation technique that involves a molecule or molecules interacting with a matrix. The matrix may take the form of solid or porous beads, resin, particles, membranes, or any other suitable material. Unless otherwise specified, chromatography includes both flow-through and batch techniques.

As used herein, the term “precipitation” refers to the process whereby one or more components present in a solution, suspension, emulsion or similar state form a solid material.

As used herein, the terms “precipitation solution” and “precipitating solution” refer to any solution, suspension, or other fluid that induces precipitation. Unless otherwise specified, a precipitation solution may also provide neutralization.

As used herein, the term “neutralization” refers to a process whereby the pH of an acidic or an alkaline material is brought near to neutrality. Typically, neutralization brings the pH into a range of about 6 to about 8.

As used herein, the terms “neutralization solution” and “neutralizing solution” refer to any solution, suspension, or other fluid which results in neutralization when mixed with an acidic or an alkaline material. Unless otherwise specified, a neutralization solution may also provide precipitation.

As used herein, the term “neutralization/precipitation solution” refers to any solution, suspension or other fluid that provides both neutralization and precipitation.

As used herein, the term “cellular components” includes any molecule, group of molecules, or portion of a molecule derived from a cell. Examples of cellular components include, but are not limited to, DNA, RNA, proteins, plasmids, lipids, carbohydrates, monosaccharides, polysaccharides, lipopolysaccharides, endotoxins, amino acids, nucleosides, nucleotides, and so on.

As used herein, the term “membrane,” as used with respect to chromatography or separations methods and materials, refers to any substantially continuous solid material having a plurality of pores or channels through which fluid can flow. A membrane may, without limitation, comprise geometries such as a flat sheet, pleated or folded layers, and cast or cross-linked porous monoliths. By contrast, when used in reference to a cell component, the term “membrane” refers to all or a part of the lipid-based envelope surrounding a cell.

As used herein, the term “bubble mixer” refers to any device that uses gas bubbles to mix two or more unmixed or incompletely mixed materials.

As used herein, the term “cell suspension” refers to any fluid comprising cells, cell aggregates, or cell fragments.

As used herein, the term “cell lysate” refers to any material comprising cells, wherein a substantial portion of the cells have become disrupted and released their internal components.

As used herein, the term “lysis solution” refers to any solution, suspension, emulsion, or other fluid that causes lysis of contacted cells.

As used herein, the term “clarified lysate” refers to a lysate that has been substantially depleted of visible particulate solids.

As used herein, the term “macroparticulate” refers to solid matter comprising particles greater than or about 100 μm in diameter.

As used herein, the term “microparticulate” refers to solid matter comprising particles less than about 100 μm in diameter.

As used herein, the terms “ultrafiltration” and “UF” refer to any technique in which a solution or a suspension is subjected to a semi-permeable membrane that retains macromolecules while allowing solvent and small solute molecules to pass through. Ultrafiltration may be used to increase the concentration of macromolecules in a solution or suspension. Unless otherwise specified, the term ultrafiltration encompasses both continuous and batch techniques.

As used herein, the terms “diafiltration” and “DF” refer to any technique in which the solvent and small solute molecules present in a solution or a suspension of macromolecules are removed by ultrafiltration and replaced with different solvent and solute molecules. Diafiltration may be used to alter the pH, ionic strength, salt composition, buffer composition, or other properties of a solution or suspension of macromolecules. Unless otherwise specified, the term diafiltration encompasses both continuous and batch techniques.

As used herein, the terms “ultrafiltration/diafiltration” and “UF/DF” refer to any technique or combination of techniques that accomplishes both ultrafiltration and diafiltration, either sequentially or simultaneously.

### DESCRIPTION

Aspects of the present invention include a lysis coil apparatus that can be integrated into an overall system or process for plasmid production or plasmid manufacturing, and in particular, for those that include large scale production of DNA plasmid. The lysis coil apparatuses described herein can be integrated into a manufacturing process such that cell suspensions that are intended to be mixed with a lysis solution, such as an alkali lysis solution, can be flowed together into one end of the lysis coil apparatus. The mixture can traverse the length of the lysis coil apparatus and then exit from the opposite end of said lysis coil apparatus into a chamber or other like-apparatus to enable neutralization of the lysis solution. Preferably, the length and time enable nearly all cells to be lysed without damaging the desired polynucleotide, for example, a DNA plasmid, while at the same time avoiding undesirable breakage of genomic polynucleotide. The lysis coil apparatus is comprised of a lysis coil holder, and a lysis coil. Preferably, the lysis coil holder is of a symmetrical geometric shape, and more preferably a cylinder. The lysis coil holder has grooves on the exterior surface capable of receiving the lysis coil. Preferably, the grooves are of a depth and size to receive and support the lysis coil, and more preferably, the grooves are symmetrically arranged throughout the height of the lysis coil holder at a desired pitch. The aforementioned allows a desired duration of time for the solution to traverse the length of the lysis coil at the linear flow rates described herein to effectively lyse the cells.

Cell cultures can be generated using a number of any one of available fermentation processes, including batch fermentation and fed-batch fermentation. One embodiment of the fermentation apparatus and processes that then lead to the cell suspension combining with a lysis solution into and through the lysis coil, are those described in US Pub. No. 2009/0004716 A1. In particularly preferred embodiments, the cells are E. coli containing a high copy number plasmid of interest, and the plasmid-containing cells are fermented to high density using batch or fed batch techniques. The cells are harvested by any means, such as centrifugation or filtration, to form a cell paste. Such harvesting methods are well known to those skilled in the art. Methods for preparing such plasmid-containing E. coli cells and performing such batch or fed-batch fermentation are well known to those skilled in the art. The cells may be harvested by routine means such as centrifugation or filtration to form a cell paste. Such harvesting methods are well known to those skilled in the art. Harvested cells may be lysed using a lysis solution to release their contents, including the biologically active molecules of interest, into a lysate solution. Furthermore, those skilled in the art will recognize that harvested cells or cell paste may be processed immediately, or stored in a frozen or refrigerated state for processing at a later date.

High yield fermentation processes are important to produce high yields of DNA plasmids, as high growth will lead to high levels of starting material. These high yield fermentation processes includes those that provide >500 mg/L plasmid yields, which include the Merck process (described in greater detail in publication WO2005078115, which is incorporated herein in its entirety), Boerhinger Ingleheim process (described in greater detail in publication WO2005097990, which is incorporated herein in its entirety), and the Nature Technology Corporation process (described in greater detail in publication WO2006023546, which is incorporated herein in its entirety), among others.

Generally, prior to lysing the cells via the lysis coil apparatus, the cell paste may be used to prepare a suspension of cells containing the biologically active molecule of interest. The cells may be suspended in any suitable solution. The suspension containing the cells in suspension solution may be maintained in a tank or other storage container. Two containers may be used wherein the second container may be used to resuspend additional amount of cells while the first container is used in the lysis process. In some embodiments, the suspension solution may comprise about 25 mM Tris-hydrochloride (“Tris-HCl”), and about 10 mM edetate disodium (“Na₂EDTA”), at a pH of about 8. In some embodiments, the cell suspension may be prepared by suspending a known weight of cell paste with a known weight of suspension buffer. For example, one part cell paste may be resuspended in about 4-10 parts of buffer, in some embodiments with about 6-8 parts of buffer. In some embodiments, the optical density of the resulting cell suspension may be about 50-80 OD₆₀₀ units. In some embodiments, it may be about 60-70 OD₆₀₀ units.

Subsequent to the fermentation process, cells can be lysed to release their contents, including the cellular components of interest, into solution. A lysis solution can be loaded into a tank, the lysis solution preferably containing one or more lysis agents, such as an alkali, an acid, an enzyme, an organic solvent, a detergent, a chaotrope, a denaturant, or a mixture of two or more such agents. More preferably, the lysis solution comprises an alkali, a detergent, or a mixture thereof. Suitable alkalis include, but are not limited to, NaOH, LiOH, or KOH. Detergents may be nonionic, cationic, anionic, or zwitterionic. Suitable detergents include, but are not limited to, sodium dodecyl sulfate (“SDS”), Triton, Tween, pluronic-type agents (block-copolymers based on ethylenoxide and propylenoxide), Brij, and CHAPS, CHAPSO, bile acid salts, cetyltrimethylammonium, N-lauroylsarcosine, and Zwittergent. Selection of suitable alkali or detergent will be well within the ordinary skill of the art. In some embodiments, the lysis solution may comprise NaOH and SDS. In some embodiments, the concentration of NaOH may be about 0.1 to about 0.3 N, and in some embodiments, about 0.2 N. In some embodiments, the concentration of SDS may be about 0.1% to about 5%, and in some embodiments about 1%. In some embodiments, the lysis solution may be maintained in a tank or other storage container. Preferred methods for performing this step are disclosed herein, and are described in detail below.

The cell suspension and lysis solution may be combined to lyse the cells and produce a lysate solution. In some embodiments, they are combined, mixed and maintained as a mixture for a time sufficient to facilitate high levels of lysis of cells and release of biological materials, thus forming the lysate solution.

In some embodiments, cell suspension and lysis solution are maintained in separate tanks and retrieved from such tanks using one or more pumps. The cell suspension and lysis solution may be brought into contact with each other using a “Y” connector, or any other connector that introduces the cell suspension with lysis solution at or near the receiving end of the lysis coil. The connector then connects to the lysis coil apparatus through a first end of the lysis coil, preferably the lower end of the lysis coil. In some embodiments, equal volumes of cell suspension and lysis solution may be pumped at equal flow rates using a dual head pump. However, those of skill in the art will recognize that cell suspension and lysis solution of different volumes may be pumped at different rates, using individual pumps, if so desired. In some embodiments, cell suspension and lysis solution are simultaneously pumped through a dual head pump, or 2 separate pumps, from about 0.3 L/min to about 2 L/min, with the contacted fluids exiting the “Y” connector at a rate from about 0.6 L/min to about 4 L/min. Those of skill in the art will recognize that these flow rates can be easily increased or decreased, and tubing size increased or decreased, to meet any throughput requirement. After exiting the “Y” connector, the cell suspension and lysis solution are flowed through, together, into a first end of the lysis coil and traverse the entire length of the lysis coil until they exit from the second end of the lysis coil.

One aspect of the present invention relates to a method for lysing cells in a controlled manner so as to extract cellular components of interest. The cells may be any cells containing cellular components of interest. Preferably, they are microbial cells. More preferably, they are E. coli cells. The present invention may be employed to extract any cellular component of interest from cells. Preferably, these will be macromolecules such as plasmids or proteins. More preferably, they are plasmids. Thus, in one preferred embodiment, the present invention relates to an advantageous method for lysing plasmid-containing E. coli cells so as to extract and eventually isolate the plasmids.

Another aspect of the present invention relates to a method for purifying cellular components of interest from a cell lysate. The cell lysate may be a lysate of any type of cells containing the cellular components of interest. Further, the cell lysate may be produced by any means known to one of skill in the art. Preferably, the lysate comprises lysed plasmid-containing cells. More preferably, the lysate comprises plasmid-containing cells lysed with alkali, detergent, or a combination thereof. Preferably, the cellular components of interest are plasmids.

In some embodiments, lysis coil apparatuses are designed to ensure consistency of the process by maintaining desired lysis coil parameters, as provided herein, while employing a single use product contact flow path. The lysis coil apparatus is designed to hold the single use lysis coil of the required internal diameter and the required length at the required angle, achieving a desired pitch, to retain solution (or solutions) for the required time at the process flow rate.

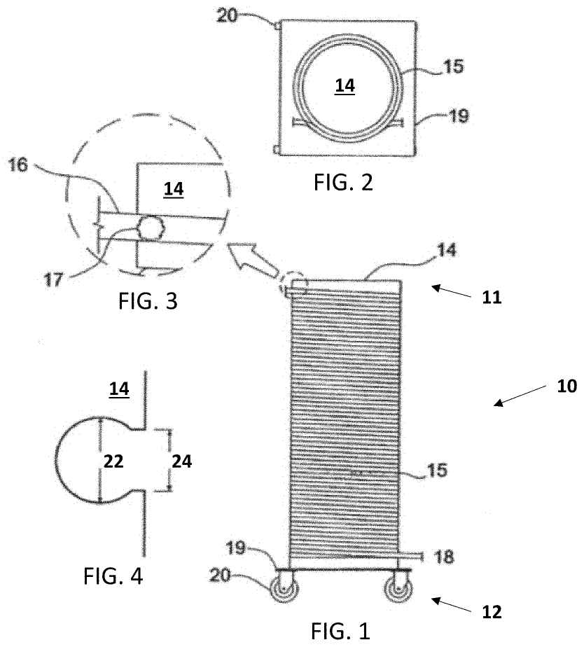

In one embodiment, a mixed combination of resuspended E. coli and lysis solution was flowed into the lower end of the lysis coil, and was retained for 5±1 minutes at the process flow rate of 2.8 L/min. There was determined to be no turbulent flow, no retention or separation of dissimilar densities of fluid. There was linear flow through the coil, and the fluid exits the top of the coil having fully denatured the E. coli cellular components. The lysis coil apparatus incorporates design elements which enable the simple and rapid installation and removal of a disposable fluid flow path which maintains the critical parameters, as provided herein. Drawings of an embodiment of the lysis coil apparatus is provided in FIGS. 1-4.

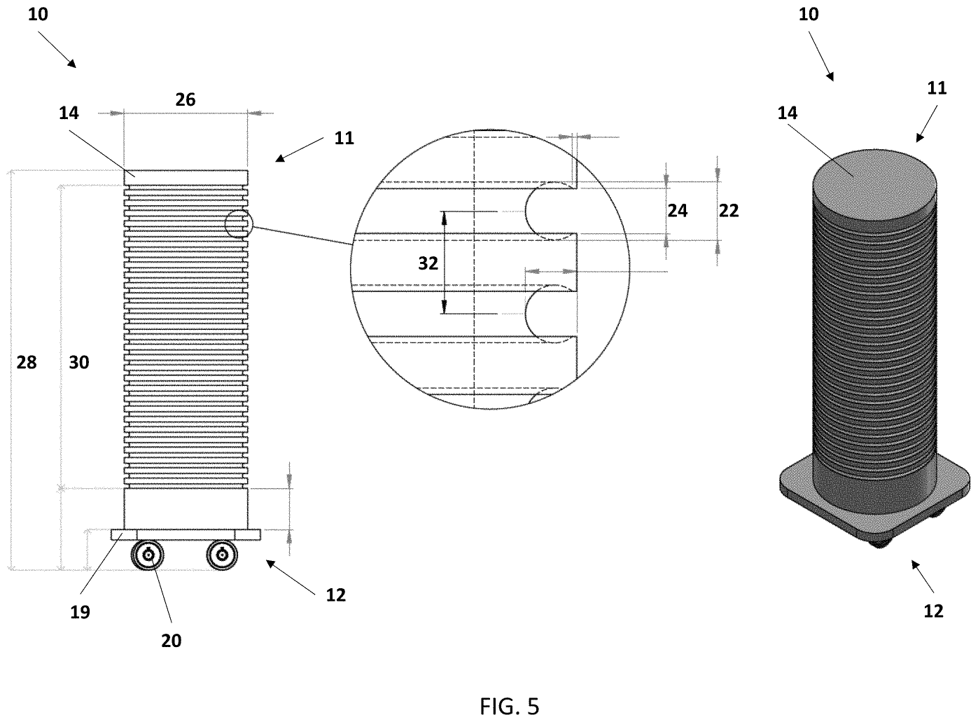

FIG. 1, FIG. 2, and FIG. 5 depict a side view, a top view, and a technical diagram, respectively, of an exemplary lysis coil apparatus 10. Apparatus 10 comprises a substantially cylindrical column 14 having a superior end 11 and an inferior end 12. Column 14 has a diameter 26 and a height 28 having any suitable dimensions. For example, diameter 26 can be about 20, 21, 22, 23, 24, 25, 26, 27, 28, 29, or 30 inches, and preferably 23, 24, or 25 inches, and height 28 can be at least 1 foot or greater than 12 feet, such as about 6 feet. In some embodiments, column 14 is stackable, such that two or more columns 14 may be combined to increase or decrease the height of apparatus 10 as desired. Column 14 can be stably attached to a base 19. In some embodiments, base 19 can further include one or more wheeled supports 20 to facilitate movement and transport of apparatus 10. Each wheeled support 20 can be lockable to park apparatus 10 in place.

Apparatus 10 further comprises lysis coil 15, an elongate tube having a lumen connecting an open first end 16 and an open second end 18. Lysis coil 15 can be coiled in a helical fashion around the exterior surface of column 14, such that a first end 16 of lysis coil 15 is positioned near the superior end 11 of column 14 and a second end 18 of lysis coil 15 is positioned near the inferior end 12 of column 14. First end 16 and second end 18 are each fluidly connectable to pumps, valves, tubing, reservoirs, containers, tanks, adapters, connectors, and the like to carry out a desired lysing process. As shown in FIG. 3, first end 16 and second end 18 can both extend freely from apparatus 10. In some embodiments, one or more retaining clips 17 can be employed to secure first end 16 and second end 18 to column 14.

As shown in FIG. 4 and FIG. 5, column 14 can include a groove 22 embedded within its exterior surface sized to fit the outer diameter of lysis coil 15. Groove 22 can be formed in a helical pattern around the exterior surface of column 14 to guide the coiling of lysis coil 15. In some embodiments, groove 22 can have a groove opening 24 smaller than the diameter of groove 22 to securely hold a lysis coil 15. Groove 22 can span a section of column 14 having height 30. The dimensions of height 30 may vary depending on overall height 28 of apparatus 10, the length of lysis coil 15, and the size of groove pitch 32 (i.e., the distance between each groove 22). For example, height 30 may be at least 6 inches or greater than 11 feet, such as about 5 feet.

The lysis coil apparatus, employed using a single use lysis retention coil, will eliminate batch to batch product carryover. The consistent retention of plasmid bearing E. coli cells combined with lysis solution for the specified amount of time enabled through the use of the principles described herein and ensured through the use of this apparatus has been demonstrated to increase plasmid yield from the lysis process by 300% and overall purification process yield by 300% as compared to the use of a larger internal diameter tubing at an uncontrolled angle.

Preferred embodiments of the lysis coil apparatus include the following properties:

### Fluid Retention Times for Preferred Embodiments:

The retention time of the mixture of cell suspension and lysis solution in the lysis coil is from 1 min to 10 min; 2 min to 10 min, 2 min to 9 min; 2 min to 8 min, 2 min to 7 min, 2 min to 6 min, 2 min to 5 min, 3 min to 10 min, 3 min to 9 min, 3 min to 8 min, 3 min to 7 min, 3 min to 6 min, 3 min to 5 min, 4 min to 10 min, 4 min to 9 min, 4 min to 8 min, 4 min to 7 min, 4 min to 6 min, 4 min to 5 min, 5 min to 10 min, 5 min to 9 min, 5 min to 8 min, 5 min to 7 min, or 5 min to 6 min. Preferably, the retention time is from 4 to 6 min; and preferably retention time is about 4.8 min, 4.9 min, 5.0 min, 5.1 min, or 5.2 min.

### Fluid Flow Rates for Preferred Embodiments:

The fluid flow rate (volume/time) of the mixture of cell suspension and lysis solution traversing through the lysis coil is a rate that achieves a linear flow rate (length/time) that results in a homogenous solution. The linear flow rate to achieve such range from about 5 m/min to about 25 m/min, 5 m/min to about 20 m/min, 5 m/min to about 19 m/min, 5 m/min to about 18 m/min, 5 m/min to about 17 m/min, 5 m/min to about 16 m/min, 5 m/min to about 15 m/min, 5 m/min to about 14 m/min, 5 m/min to about 13 m/min, 5 m/min to about 12 m/min, 5 m/min to about 11 m/min, 5 m/min to about 10 m/min, 6 m/min to about 25 m/min, 6 m/min to about 20 m/min, 6 m/min to about 19 m/min, 6 m/min to about 18 m/min, 6 m/min to about 17 m/min, 6 m/min to about 16 m/min, 6 m/min to about 15 m/min, 6 m/min to about 14 m/min, 6 m/min to about 13 m/min, 7 m/min to about 12 m/min, 6 m/min to about 11 m/min, 6 m/min to about 10 m/min, 7 m/min to about 25 m/min, 7 m/min to about 20 m/min, 7 m/min to about 19 m/min, 7 m/min to about 18 m/min, 7 m/min to about 17 m/min, 7 m/min to about 16 m/min, 7 m/min to about 15 m/min, 7 m/min to about 14 m/min, 7 m/min to about 13 m/min, 7 m/min to about 12 m/min, 7 m/min to about 11 m/min, 7 m/min to about 10 m/min, 8 m/min to about 25 m/min, 8 m/min to about 20 m/min, 8 m/min to about 19 m/min, 8 m/min to about 18 m/min, 8 m/min to about 17 m/min, 8 m/min to about 16 m/min, 8 m/min to about 15 m/min, 8 m/min to about 14 m/min, 8 m/min to about 13 m/min, 8 m/min to about 12 m/min, 8 m/min to about 11 m/min, 8 m/min to about 10 m/min, 9 m/min to about 25 m/min, 9 m/min to about 20 m/min, 9 m/min to about 19 m/min, 9 m/min to about 18 m/min, 9 m/min to about 17 m/min, 9 m/min to about 16 m/min, 9 m/min to about 15 m/min, 9 m/min to about 14 m/min, 9 m/min to about 13 m/min, 9 m/min to about 12 m/min, 9 m/min to about 11 m/min, or 9 m/min to about 10 m/min; and more preferably 8 m/min to 10 m/min; and includes embodiments of about 8 m/min, 8.25 m/min, 8.50 m/min, 8.75 m/min, 9 m/min, 9.25 m/min, 9.50 m/min, 9.55 m/min, 9.60 m/min, 9.65 m/min, 9.70 m/min, 9.75 m/min, 9.80 m/min, 9.85 m/min, 9.90 m/min, 9.95 m/min, and 10 m/min, and preferably is 9.50 m/min, 9.70 m/min, 9.75 m/min, 9.80 m/min, or 10 m/min.

Such linear flow rates incorporated into embodiments of the lysis coils with the interior diameters described herein can achieve flow rates of 5000 mL/min, 4000 mL/min, 3000 mL/min, 2900 mL/min, 2800 mL/min, 2700 mL/min, 2600 mL/min, 2500 mL/min, 2400 mL/min, 2300 mL/min, 2200 mL/min, 2100 mL/min, 2000 mL/min, 1900 mL/min, 1800 mL/min, 1700 mL/min, 1600 mL/min, 1500 mL/min, 1400 mL/min, 1300 mL/min, 1200 mL/min, 1100 mL/min, 1000 mL/min, 900 mL/min, 800 mL/min, 700 mL/min, 600 mL/min, or 500 mL/min. The flow rate is influenced by the interior diameter of the lysis coil, and the flow rates used with the lysis coil apparatuses are those used with lysis coils with the interior diameters described herein. For example, a lysis coil having an interior diameter of ¾ inch can have an overall fluid flow rate of between about 2317 mL/min and 3475 mL/min, and preferably about 2780 mL/min. In another example, a lysis coil having an interior diameter of ⅜ inch can have an overall fluid flow rate of between about 543 mL/min and 814 mL/min, and preferably about 651.5 mL/min.

### Length of Lysis Coils Used in the Preferred Embodiments:

In some embodiments the lysis coil has a length of over 100 feet, over 105 feet, over 110 feet, over 115 feet, over 120 feet, over 125 feet, over 130 feet, over 135 feet, over 140 feet, over 145 feet, over 150 feet, over 155 feet, over 160 feet, over 165 feet, over 170 feet, over 175 feet, over 180 feet, over 185 feet, over 190 feet, over 195 feet, or over 200 feet. Preferably the length is 150 feet, 155 feet, or 160 feet long.

### Height of Lysis Coils Used in the Preferred Embodiments:

In some embodiments, the lysis coil has a height between about 3 feet to about 6.25 feet, about 3.5 feet to about 6.25 feet, about 4 feet to about 6.25 feet, about 4.5 feet to about 6.25 feet, about 5.0 feet to about 6.25 feet, about 3.5 feet to about 6.0 feet, about 4 feet to about 6.0 feet, about 4.5 feet to about 6.0 feet, about 5.0 feet to about 6.0 feet, about 3.5 feet to about 5.75 feet, about 3.75 feet to about 5.75 feet, about 4 feet to about 5.75 feet, about 4.5 feet to about 5.75 feet, or about 5.0 feet to about 5.75 feet. Preferably, the height is about 5.8, 5.9, 6.0, 6.1, or 6.2 feet.

Pitch of lysis coils used in the preferred embodiments:

In some embodiments the lysis coil is aligned in the grooves of the lysis coil holder that causes the lysis coil to maintain a pitch of between 2.0 degree to 4.0 degree angle, 2.0 degree to 3.8 degree angle, 2.0 degree to 3.6 degree angle, 2.0 degree to 3.43 degree angle, 2.0 degree to 3.4 degree angle, 2.0 degree to 3.2 degree angle, 2.0 degree to 3.2 degree angle, 2.0 degree to 3.0 degree angle, 2.0 degree to 2.8 degree angle, 2.0 degree to 2.6 degree angle, 2.0 degree to 2.5 degree angle, 2.1 degree to 3.4 degree angle, 2.1 degree to 3.2 degree angle, 2.1 degree to 3.2 degree angle, 2.1 degree to 3.0 degree angle, 2.1 degree to 2.8 degree angle, 2.1 degree to 2.6 degree angle, 2.1 degree to 2.5 degree angle, 2.1 to 2.25 degree angle, 2.15 to 2.2 degree angle, or 2.1 to 2.15 degree angle, and preferably a 2.1, 2.15, or 2.2 degree angle.

### Interior Diameter of Lysis Coils Used in the Preferred Embodiments:

In some embodiments the interior diameter of the lysis coil is less than about 1 inch, including ⅞, ¾, ⅝, ½, ⅜, or ¼ inch. Preferably, the interior diameter of said lysis coil is ¾ inch in some embodiments, ½ inch in some embodiments, or ⅜ inch in other preferred embodiments.

The components of the lysis coil apparatus can be made using any suitable material. Certain components such as column 14 and base 19 can be made from a rigid material such as a plastic, a metal, or wood. Components substantially comprising a metal may be milled from a larger block of metal or may be cast from molten metal. Likewise, components substantially comprising a plastic or polymer may be milled from a larger block, cast, or injection molded. In some embodiments, the components may be made using 3D printing or other additive manufacturing techniques commonly used in the art, including but not limited to fused deposition, stereolithography, sintering, digital light processing, selective laser melting, electron beam melting, and laminated object manufacturing. The components may be individually printed or at least partially printed together to minimize assembly. Any number of materials compatible with additive manufacturing can be used, such as various polymers, including silicone and ABS; metals, including aluminum, stainless steel, and titanium; and other materials, including ceramics and composites.

Certain components such as lysis coil 15 can be made from a substantially flexible material, such as a soft or flexible polymer. Preferably the material is compatible with 0.1-1N alkali solution, preferably a USP class VI material. In some embodiments, the material is compatible with 0.5 N NaOH solution. In various embodiments, the polymers can be bioinert and resist corrosion and degradation in the presence of lysing solutions. Suitable polymers include but are not limited to including but not limited to: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO), polyorthoesters, polyvinyl chloride (PVC) and the like. The flexibility of lysis coil 15 can be modified based on its construction. For example, increasing the wall thickness of lysis coil 15 can decrease its flexibility, while decreasing the wall thickness of lysis coil 15 can increase its flexibility. In another example, the wall of lysis coil 15 can include corrugation to enhance flexibility, wherein the corrugation can be featured on the exterior of lysis coil 15 so as to not disturb fluid flow within.

In certain embodiments, lysis coil 15 can be modified with one or more coatings or surface treatments. The coatings or surface treatments may enhance the flow of fluids or lysing and separation of materials by altering the hydrophobicity or hydrophilicity of the inner surface of lysis coil 15. The coatings or surface treatments can be deposited or applied using any suitable means, including spin coating, dip coating, chemical vapor deposition, chemical solution deposition, physical vapor deposition, liquid bath immersion, and the like. The coatings or surface treatments can be deposited or applied with any suitable thickness.

In some embodiments, lysis coil 15 is removable from apparatus 10. For example, lysis coil 15 can be a disposable component that can be discarded and replaced with each use. Lysis coil 15 can also be provided in several different configurations having varying inner dimensions and material construction, wherein a variety of lysis coil 15 configurations can be interchanged based on the desired lysing process.

It should be understood that lysis coil apparatus 10 is amenable to any suitable modification to augment its function. For example, in certain embodiments, column 14 can house one or more devices within its interior. Suitable devices include but are not limited to: heaters, coolers, flow sensors, temperature sensors, oscillators, and the like. In another example, column 14 is rotatable about base 19 to facilitate coiling and uncoiling lysis coil 15 onto apparatus 10. Column 14 can be manually rotated, such as by way of a handle attached to its superior end 11, or column 14 can be fitted with a motor for mechanized rotation. Column 14 can further comprise a locking mechanism to arrest rotation once lysis coil 15 has been fully coiled or uncoiled.

The lysate solution resulting from the lysis coil apparatus can then be neutralized by combining it with a neutralizing solution (which is also referred to as a neutralizing precipitation solution) to produce a dispersion comprising neutralized lysate solution and debris. The resultant dispersion may then be maintained to facilitate separation of the neutralized lysate solution from the debris.

In some embodiments, lysate solution, which comprises the lysed cells, may be neutralized by mixing it with neutralizing solution in a neutralizing chamber. This neutralization of lysate solution can be facilitated by mixing in the neutralizing chamber. In some embodiments, this neutralizing can be followed by bubble mixing in a bubble column mixer. Preferably, the neutralization occurs in conjunction with bubble mixing in a bubble column mixer. In some embodiments, the lysate solution exiting the holding coil may enter a bubble column mixer while simultaneously a pump may deliver a neutralization/precipitation solution from another tank into the bubble column mixer. In some examples, also simultaneously, compressed gas from another tank may be sparged into the bottom of the bubble column mixer. In some embodiments, lysate solution may enter the column at the bottom from one side, while neutralization/precipitation solution may enter at the bottom from the opposite side. Compressed gas may be sparged in through a sintered sparger designed to deliver gas bubbles substantially uniformly across the column cross section. Lysate solution, which comprises the lysed cells, and neutralization solution flow vertically up the column and exit through an outlet port on the side near the top. The passage of the gas bubbles through the vertical column of liquid serves to mix the lysate solution with the neutralization/precipitation solution. The mixing provided by the rising gas bubbles is thorough but gentle and low shear. As the neutralization/precipitation solution mixes with the lysed cells of the lysate solution, cell components precipitate from the solution. A snorkel may be provided at the top of the bubble column mixer to vent excess gas. Example are provided in detail in co-owned patent application US Publication no. 20090004716A1, which is incorporate herein in its entirety.

In some embodiments, the lysate solution comprises plasmid-containing cells lysed with an alkali, a detergent, or a mixture thereof, and the neutralizing/precipitating solution neutralizes the alkali and precipitates host cell components such as proteins, membranes, endotoxins, and genomic DNA. In some embodiments, the alkali may be NaOH, the detergent may be SDS, and the neutralization/precipitation solution may comprise potassium acetate, ammonium acetate, or a mixture thereof. In some embodiments, the neutralization/precipitation solution may comprise an unbuffered solution containing about 1 M potassium acetate and from about 3 M to about 7 M ammonium acetate. Using such a neutralization/precipitation solution produces a suspension with a pH of from about 7 to about 8, which is preferable to an acidic pH because acidic conditions can lead to depurination of DNA. In some embodiments, the neutralization/precipitation solution may be provided in a chilled form from about 2° C. to about 8° C.

The bubble column mixer provides mixing in a low shear manner and thus avoids excessive release of genomic DNA and endotoxins into the neutralized lysate solution. One skilled in the art will be able to determine suitable rates for flowing gas through the bubble column mixer. Gas flow rates may be used at about 2 standard liters per minute to about 20 standard liters per minute (“slpm”). Any suitable gas may be used, including, but not limited to, air, nitrogen, argon, and carbon dioxide. The gas may be filtered compressed air.

The combination of lysate solution and neutralization solution results in the generation of a dispersion containing neutralized lysate solution and debris. The neutralized lysate solution may be collected in a tank or other storage container. In some embodiments, the container is chilled to 5-10° C. The time for the holding of neutralized lysate in the container is not mandatory, and may vary from less than 1 hour, from about 1 hour to about 12 hours, from about 12 hours to about 15 hours, or greater than 15 hours. In some embodiments, the time used is about 12 hours, while some examples involve a time of about 15 hours, while in other examples the time is “overnight” (defined as being greater than about 15 hours). In one embodiment, a sufficient hold period was employed to achieve substantially complete separation of the cell debris from the neutralized lysate solution, resulting in the obtained crude lysate of limited solid particles advantageous for subsequent clarification process. However, the process scale is limited to the crude lysate holding tank and the process time is elongated by this hold period.

In order to achieve large scale purification of low yield plasmid product, the period for the holding of neutralized lysate may be reduced to lower than 1 hour. In some embodiments, the neutralized lysate solution may be simultaneously processed at the time it is generated, thus the holding time in the container is negligible. In some embodiments, the lysate solution is simultaneously processed by the following process after a period from about 5 minutes to about 60 minutes of collecting the lysate in the container. The reduction or elimination of lysate holding time also removes the process capacity limit by containers as the lysate is processed immediately at its generation.

After neutralization, the neutralized lysate solution may be clarified with any approaches of solid/liquid separation, e.g. bag filtration, cartridge filtration, batch centrifugation, continuous centrifugation. Complete removal of the particles in the solution is desirable to avoid the clogging of membrane or column in the following purification processes. At the same time, the lysate may not be subjected to excessive shear that will shred genomic DNA and cause the release of genomic DNA, shredded genomic DNA, endotoxin and other contaminants into the plasmid-containing solution. Batch filtration may be used for processing small volumes of lysate, but is impractical at large scale. Continuous centrifugation is also unsuitable because the precipitate may be subjected to high shear stress and release high level of contaminant to solution. In some embodiments a series of filtrations employing different grade of filter media can be utilized. The primary filtration can be used to remove a majority of large cell flocs range in micron sizes, while the consecutive secondary filtration retains the remaining fine particles. An optional third filtration may be conducted when a stringent clarity is desired for the following process and the secondary filtration is insufficient.

Following separation of the clarified lysate, solutions containing the cellular components of interest can be subjected to ion exchange chromatography in some embodiments. Preferably, this is performed using a membrane-based approach. Preferably, this is anion exchange membrane chromatography. Specific methods for performing this step are further disclosed in detail elsewhere herein.

After ion exchange chromatography, the partially purified material resulting from ion exchange chromatography is subjected to hydrophobic interaction chromatography. Preferably, this is performed using a membrane-based approach. Specific methods for performing this step are further disclosed in detail below. In certain embodiments, this step may be omitted.

Thereafter, the material resulting from hydrophobic interaction chromatography (if performed) or from ion exchange chromatography (if HIC is omitted) is subjected to ultrafiltration and diafiltration to concentrate the cellular components of interest and to remove excess salts from the solution. Use of ultrafiltration/diafiltration is well known to those of skill in the art, especially for biological macromolecules such as proteins or plasmids.

Following the filtration steps, in some embodiments the concentrated and desalted product is optionally subjected to sterile filtration, for example to render it suitable for pharmaceutical uses. Again, methods for performing this step are well within the knowledge of those skilled in the art.

Concentrated, desalted product may, if desired, be further subjected to sterile filtration. Various methods for performing such an operation are well known, and will be within the capability of those skilled in the art. Where the product is a plasmid, sterile filtration may preferably be performed using a Pall AcroPak 200 filter with a 0.22 um cut-off. The resulting purified, concentrated, desalted, sterile-filtered plasmid is substantially free of impurities such as protein, genomic DNA, RNA, and endotoxin. Residual protein, as determined by bicinchoninic acid assay (Pierce Biotechnology, Rockford, Ill.) will preferably be less than about 1% (by weight), and more preferably less than or equal to about 0.1%. Residual endotoxin, as determined by limulus amebocyte lysate (“LAL”) assay, will preferably be less than about 100 endotoxin units per milligram of plasmid (EU/mg). More preferably, endotoxin will be less than about 50 EU/mg, most preferably less than about 20 EU/mg. Residual RNA is preferably less than or about 5% by weight, more preferably less than or about 1% (by agarose gel electrophoresis or hydrophobic interaction HPLC). Residual genomic DNA is preferably less than about 5% by weight, more preferably less than about 1% (by agarose gel electrophoresis or slot blot).

One skilled in the art will recognize that the present invention may be modified by adding, subtracting, or substituting selected steps or methods around the lysis coil apparatus, including those known or available in the art but not explicitly mentioned herein. All such modifications are contemplated to be part of the present invention. Thus, in one embodiment, the invention provides for methods of mixing a cell lysate, or a fluid containing cellular components of interest with one or more additional fluids using a bubble mixer. In a further embodiment, the invention provides for mixing a cell lysate with a precipitating solution using a bubble mixer, while simultaneously entrapping gas bubbles in the precipitated cellular components. In yet another embodiment, the present invention provides for a device comprising a bubble mixer that may be used to practice the above methods. Still further, the present invention provides for methods of lysing cells, comprising a combination of mixing a cell suspension with a lysis solution using the provided lysis coil apparatus, followed by mixing the lysed cells with a precipitating solution using a bubble mixer. In another embodiment, the invention provides for a method to separate precipitated cellular components from a fluid lysate, comprising entrapping gas bubbles in the precipitated cellular components using a bubble mixer, collecting the materials in a tank, allowing the precipitated cellular components to form a floating layer, optionally applying a vacuum to compact the precipitated components and degas the lysate, and recovering the fluid lysate by draining or pumping it out from underneath the precipitated components. In yet another embodiment, the present invention provides a method for purifying cellular components of interest from a cell lysate, comprising subjecting the lysate to an ion exchange membrane, optionally a hydrophobic interaction membrane, an ultrafiltration/diafiltration procedure, and optionally, a sterile filtration procedure. Each of the current embodiments, as well as any combination of one or more embodiments, is further encompassed by the present invention.

The innovative teachings of the present invention are described with particular reference to the steps disclosed herein with respect to the production of plasmids. However, it should be understood and appreciated by those skilled in the art that the use of these steps and processes with respect to the production of plasmids provides only one example of the many advantageous uses and innovative teachings herein. Various non-substantive alterations, modifications and substitutions can be made to the disclosed process without departing in any way from the spirit and scope of the invention. The following examples are provided to illustrate the methods and devices disclosed herein, and should in no way be construed as limiting the scope of the present invention.

### Example 1: Lysis Coil Internal Diameter (ID) and Angle of Installation were Determined to have an Impact on Overall Process Yield

Separate tests indicated that a ¾″ ID coil provided more homogenous linear flow via a faster linear velocity as compared to a 1″ ID coil at equal process flow rates.

Another set of tests indicated that flow rates scaled to the same linear velocity with a 1″ ID coil, the ¾″ ID coil displayed more homogenous linear flow. This demonstrates the optimal maximum ID for the lysis coil is ¾″ and the hold time of 5+/−1 minutes should be adjusted for faster flow rates by increasing or decreasing the length of the coil. Processing at reduced flow rates may be accomplished using a lysis coil with a smaller diameter and equal length with conserved linear velocity.

To determine the optimal angle of the coil the 1″ ID and ¾″ ID coils were tested at a 3′ height and 6′ height on a 24″ diameter holder. Both the 1″ and ¾″ ID coils performed better at the 6′ total height with angles at 3.43° and 2.15° respectively. The ¾″ ID coil demonstrated more homogenous linear flow of the crude lysate through the coil.

These sets of tests indicated that a ¾″ ID coil at an angle of 2.15-3.43° would result in the most optimal performance of the lysis coil.

As a single use lysis coil is desired, a prototype holder was designed for fast, simple, and consistent installation of a ¾″ ID single use lysis coil, with the length required to obtain a 4-6 minute retention time, and the angle necessary to facilitate homogenous linear flow. The prototype was successfully used for multiple production lots at varying production scales.

The prototype was further developed into the current design. A polypropylene grooved cylinder designed to hold a 160 foot length of ¾″ ID tubing at a 2.15° angle. This design facilitates faster and more consistent installation of the lysis coil than the prototype while maintaining the desired linear velocity and homogenous flow of crude lysate through the coil.

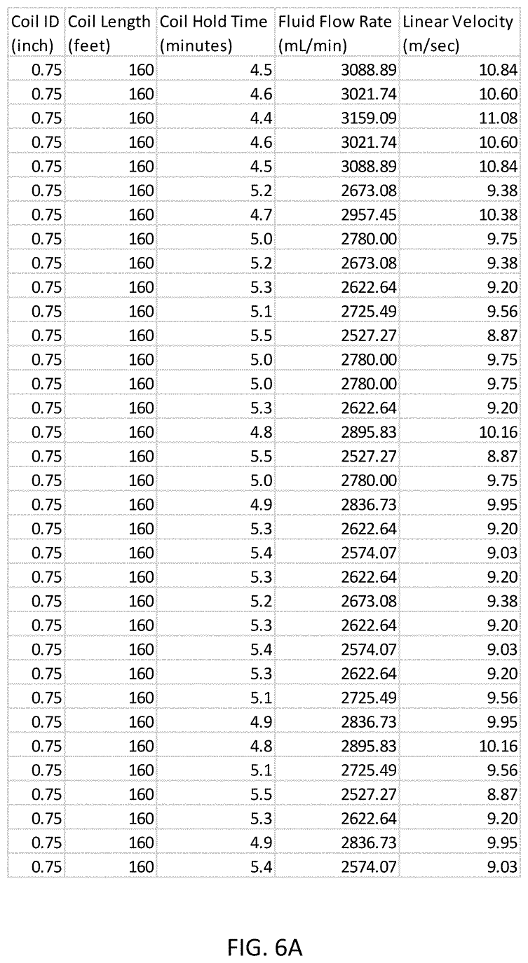

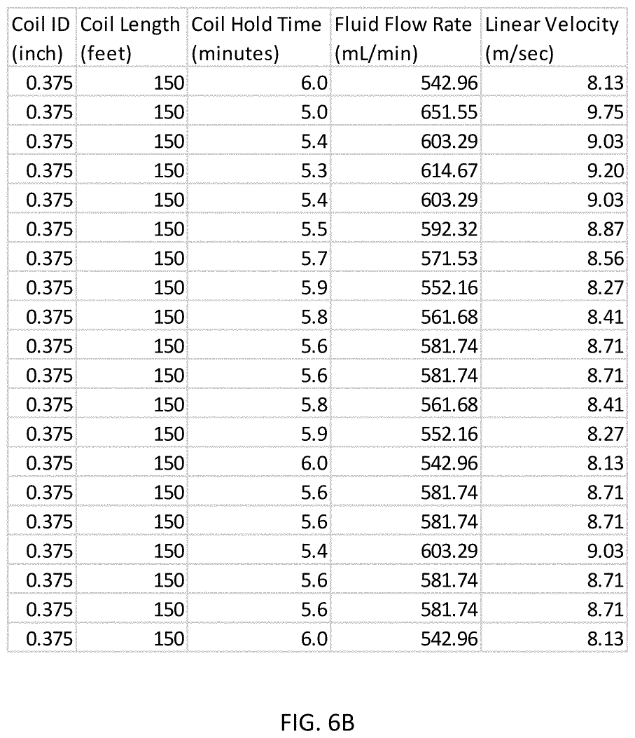

A further set of tests investigated the relationship between coil hold time, fluid flow rate, and fluid linear velocity in two lysis coils, the first coil with a 160 foot length of ¾ inch ID tubing and the second coil with a 150 foot length of ⅜ inch ID tubing. The results are shown in FIG. 6A and FIG. 6B, respectively.

### Example 2: Process of DNA Plasmid Manufacturing

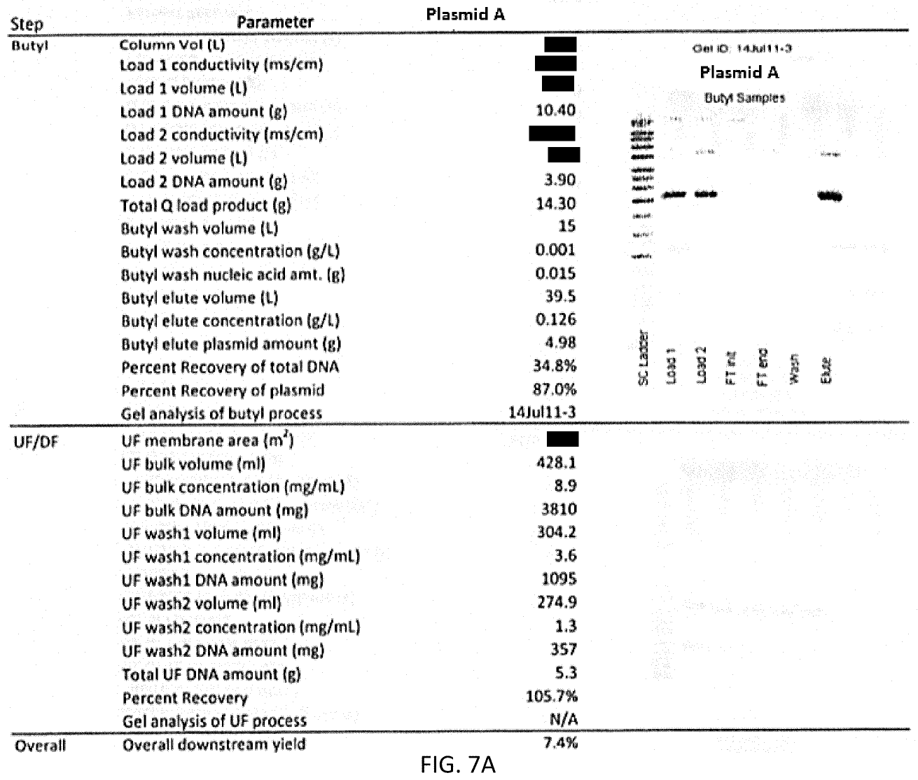

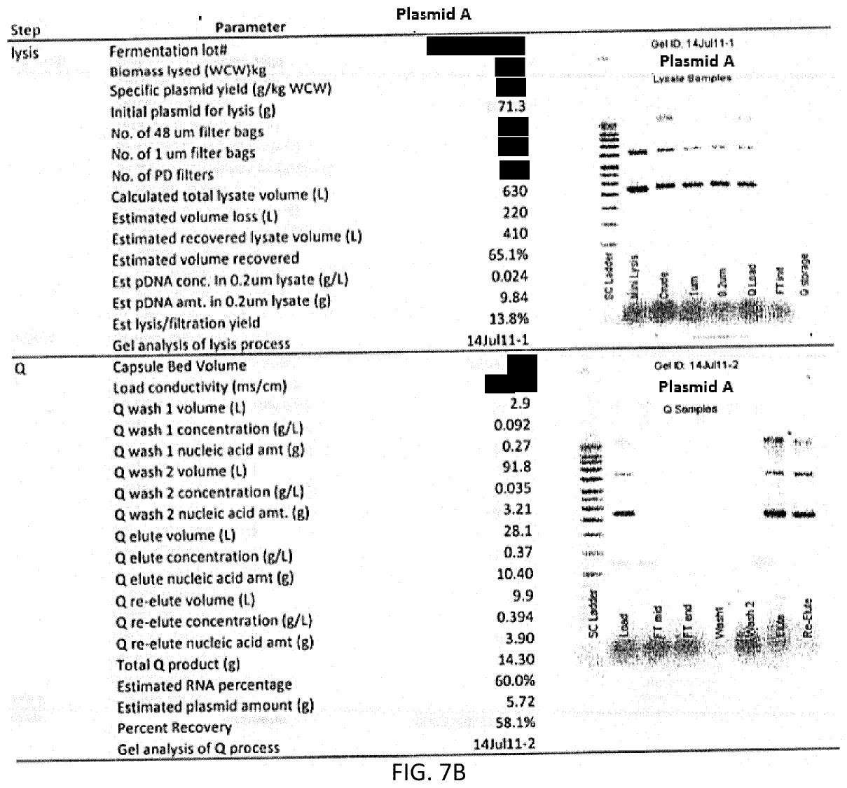

Process for DNA plasmid manufacturing from a 400 L fermentation batch included: i) cell lysis and filtration; ii) Mustang Q anion exchange membrane chromatography; iii) butyl hydrophobic interaction chromatography; and iv) ultrafiltration/diafiltration (UF/DF). Purification data for Plasmid A is summarized in FIG. 7A and FIG. 7B.

Initial DNA plasmid in the cell paste was estimated at 3.17 g/kg WCW (wet cell weight) by miniprep method, and initial plasmid before purification was 71.3 g. Final UF product was 5.3 g, resulting in a 7.4% overall purification yield. This result was determined to be an atypical yield for a 400 L fermentation batch.

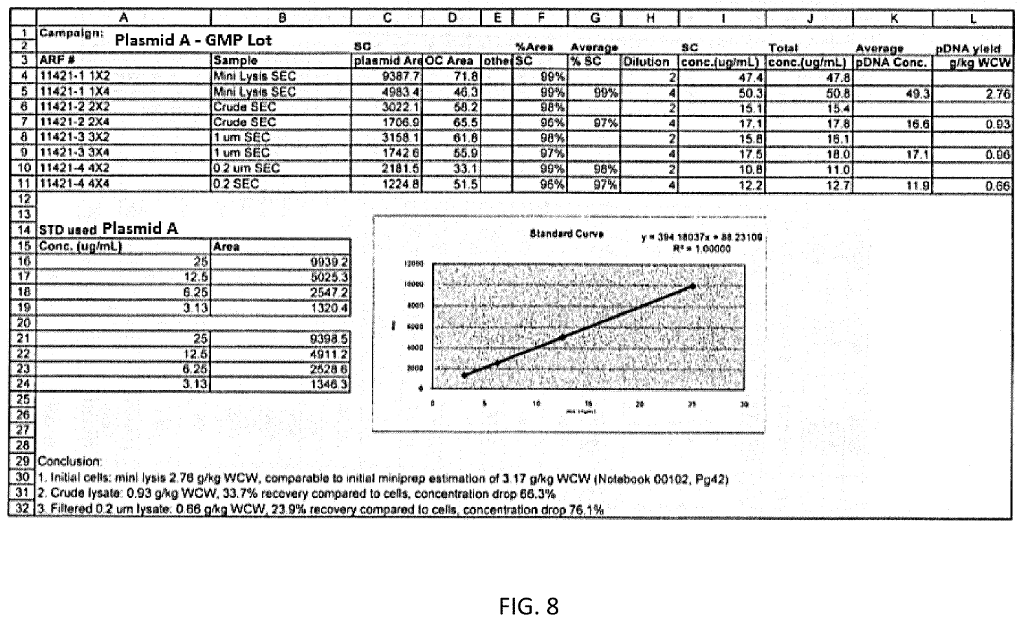

Yield analysis was conducted for each step (see FIG. 7A and FIG. 7B). UF (iv) had a step yield of >100%, therefore step iv was excluded as the cause of low yield. Butyl step (iii) had a recovery of 34.8% for total DNA, which appeared lower than a typical 60% recovery. However, gel 14Jul11-4 (FIG. 7A and FIG. 7B) indicated that percentage of open circular (OC) and gDNA were removed in the flow-through due to 1:4 v/v load dilution with 3M ammonium sulfate. There was no evidence to suggest loss of supercoiled plasmid product in the butyl step. Taken into consideration of ˜60% RNA in the butyl load, the recovery of plasmid was 87.0%. Therefore, step iii was not the origin of the reduced yield. Q step (ii) obtained 14.3 g total DNA, but initial material before Q was estimated at 10.4 g total DNA based on HPLC analysis of Plasmid A crude lysate (FIG. 8). Estimated 5.7 g plasmid in the Q product contributed to ˜58.1% of Q step recovery of plasmid (comparable to ˜50% Q recovery of a 5 kb plasmid), showing that the performance of step ii was typical. As step ii, iii, and iv are excluded, step I was concluded to be the phase primarily responsible for the reduced purification yield.

As in FIG. 8, HPLC analysis of resuspended cells and different stages of lysate demonstrated an apparent concentration drop between cells and crude lysate samples. Resuspended cells still had a yield of 2.8 g/kg WCW, comparable to initial estimation of 3.17 g/kg. But crude lysate had a yield of 0.9 g/kg WCW, which was 68% lower in concentration. Estimated 35% of volume reduction from filtration added up to 86% plasmid loss in Step i. Combined lysis/filtration recovery was only 13.8%. The mass balance concluded that initial lysis phase (and not filtration) was the root cause of the Plasmid A low yield.

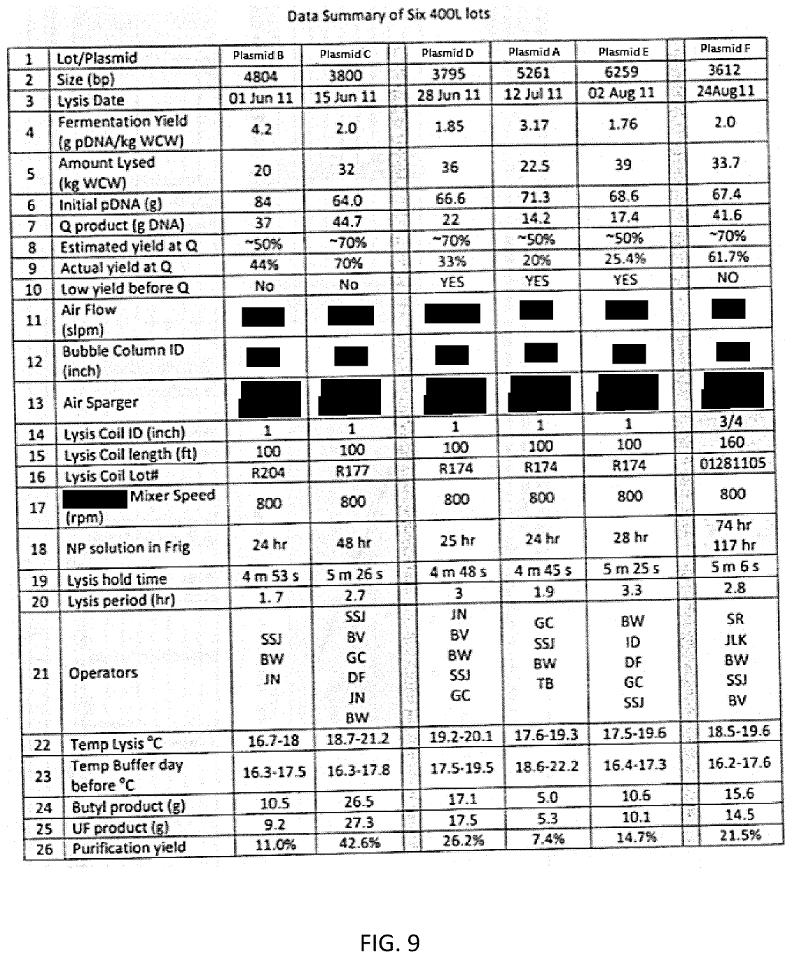

Possible factors contributing to the process of cell lysis were studied, including a) air flow, b) bubble column inner diameter, c) air sparger, d) lysis coil, e) mixer, f) solution, g) operators, and h) room temperature. Data from 6 separate plasmid production lots (Plasmid B, C, D, A, E, and F) were reviewed (See FIG. 9). Factors a, b, c, and e were concluded as having minimum impact when they were within specifications. Factor f was deemed a minimal factor, and eliminated altogether when ambient temperature is less than or equal to 25 degree C. Factor g did not suggest any trends relative to human operators, and thus not the cause of the low yields. Factor h of room temperature may contribute to solution storage variability, but no clear differences were identified for one plasmid purification compared to the other purification lots.

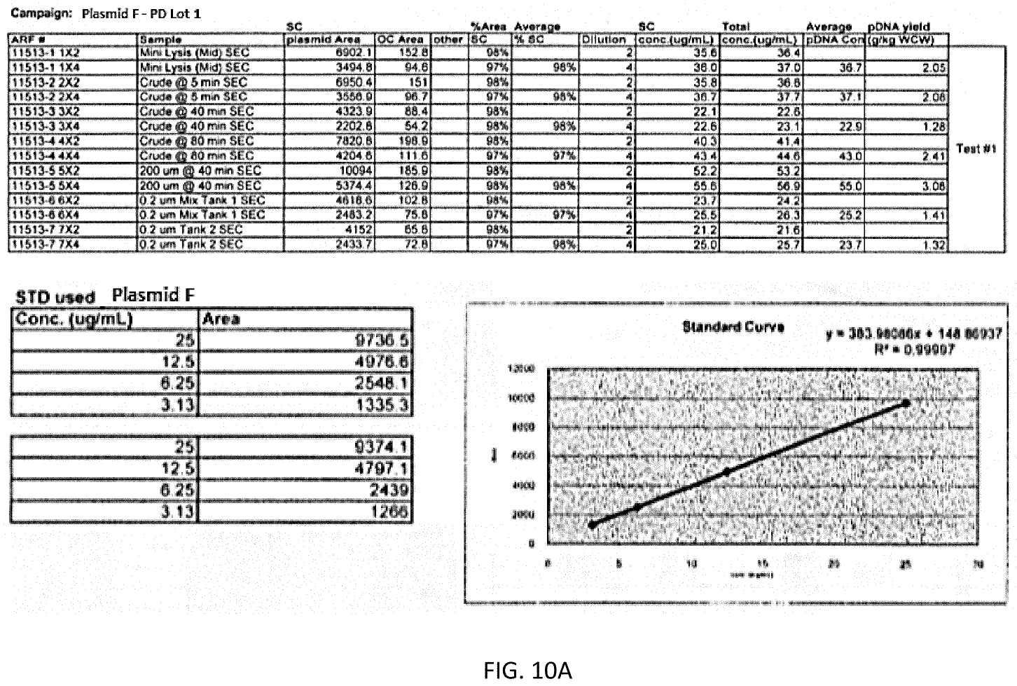

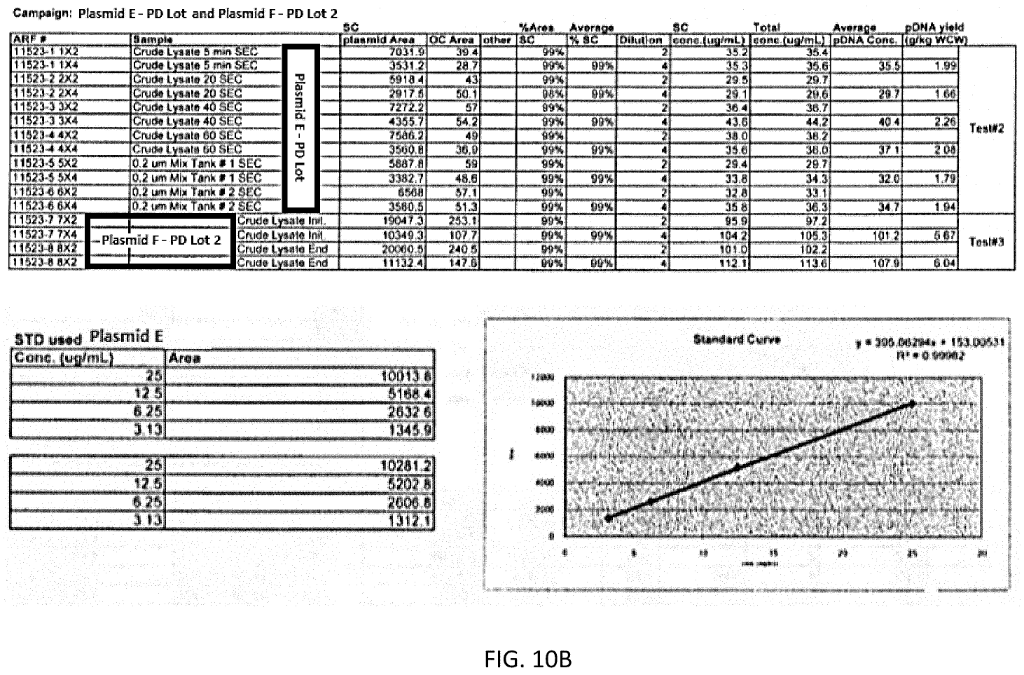

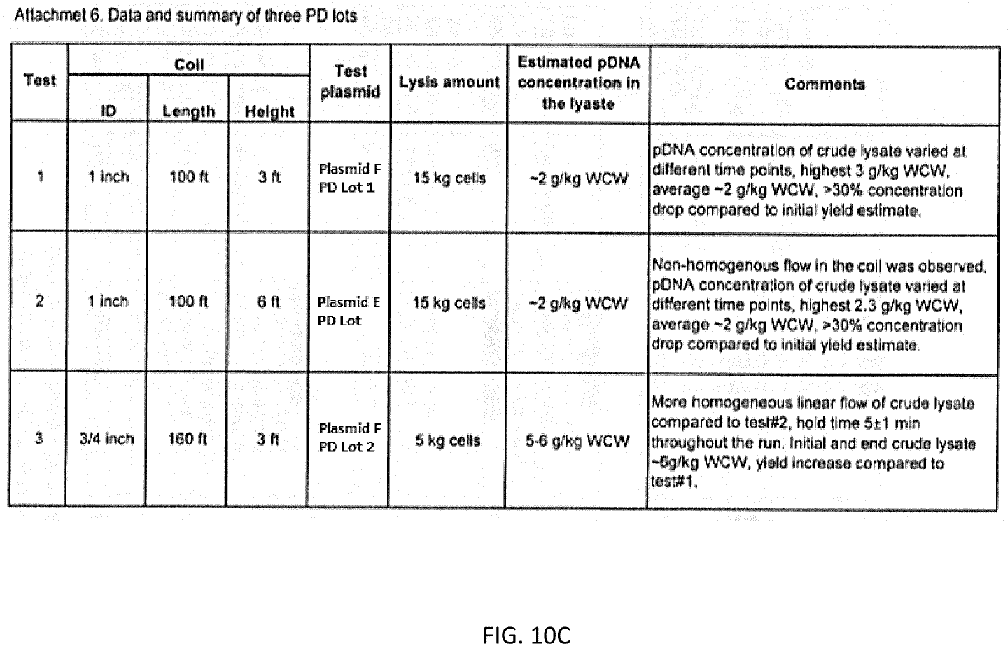

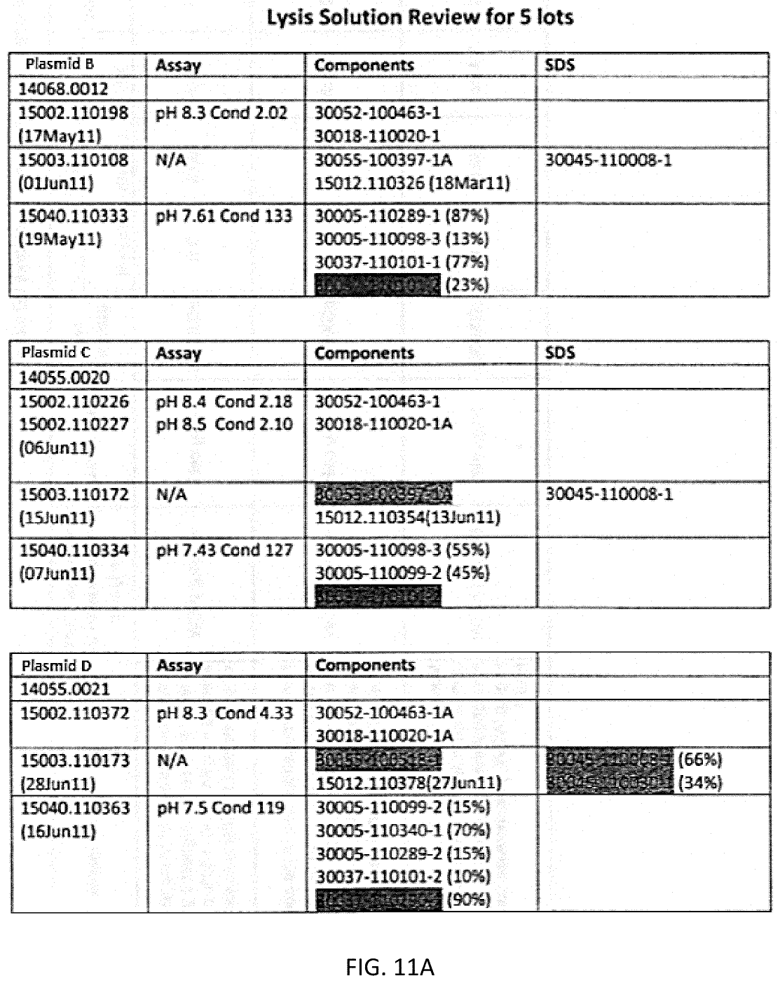

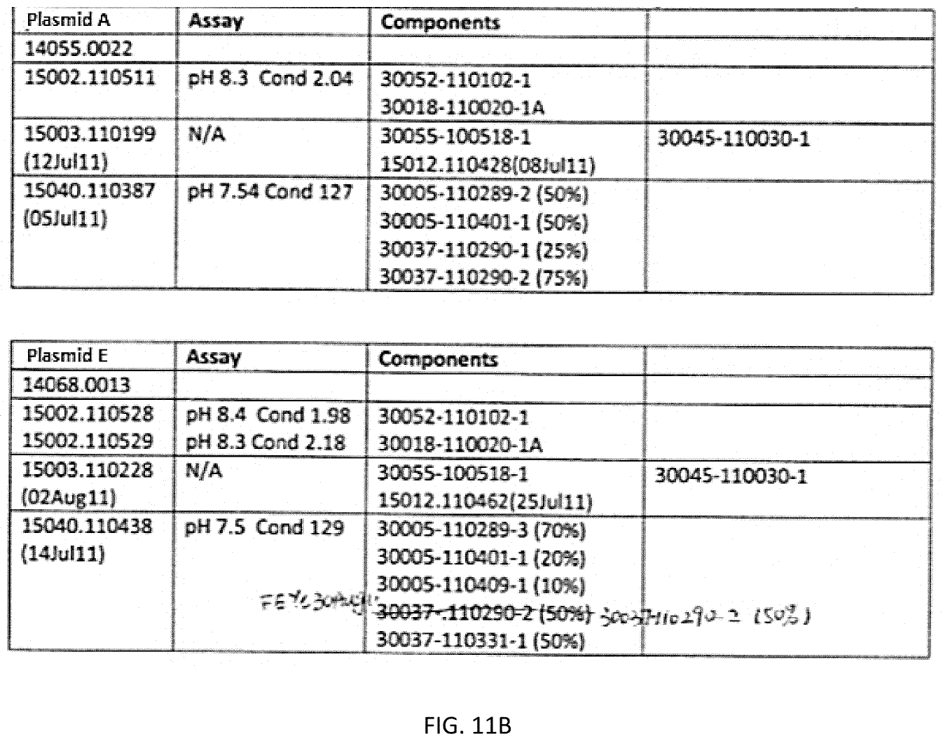

The most possible cause leading to low lysis yield was suspected to be factor d-lysis coil. Prior to a production run for Plasmid E and Plasmid F, the lysis coil was cleaned, sanitized and re-used. The re-assembly of a new coil was implemented for these plasmids. Variation in coil angle and height varied by different operators, which could impact the flow homogeneity of crude lysate during different runs. Crude lysate hold up time of 5+/−1 min is critical for cell integration and plasmid renaturing afterwards. Three production design tests with different coils at different lengths and angles were conducted. Data and conclusions are provided in FIG. 10A through FIG. 10C. More homogeneous linear flow of crude lysate was observed in test #3 using a smaller interior diameter coil—¾ inch interior diameter and 160 feet long, compared to test #2—1 inch interior diameter and 100 feet long. Hold time of 5+/1 min was confirmed throughout the run in test #3, but suspected to be inconsistent for 1 inch interior diameter lysis coils from the >30% variation of crude lysate concentrations. Yield increase was demonstrated for ¾ inch interior diameter lysis coils, and production runs with Plasmid F were planned with such a coil.

### Example 3: Purification of Plasmid

The purification of Plasmid F was performed using a lysis coil with an internal diameter (ID) of ¾″ and made of polyvinyl chloride (PVC) (Thermoplastic Processes). The PVC tubing was in compliance with USP class VI and manufactured in accordance with 21 CFR 178.3740. Additionally, HDPE was chosen for the connectors used in the manufacturing process. The HDPE connectors are distributed by Cole Parmer and are a USP class VI material and manufactured in accordance with 21 CFR 177.1520. The lysis coil was long enough to generate a lysis hold time of 5+/−1 minutes with stainless steel ½″ barbed fittings on each end. This resulted in the lysis coil having a length of 160 feet.

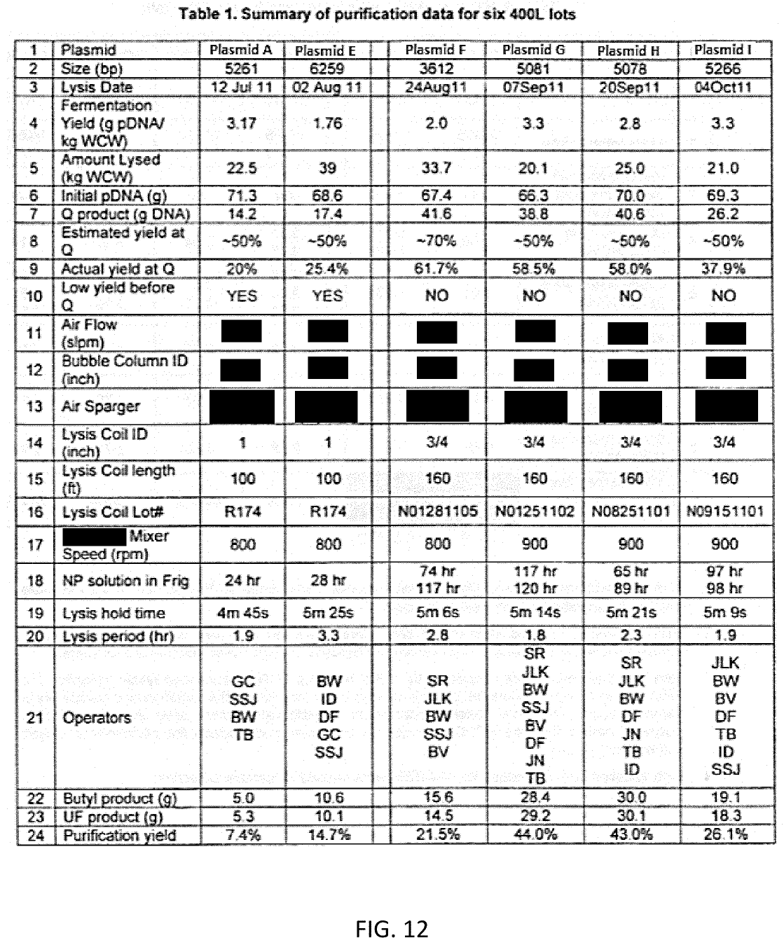

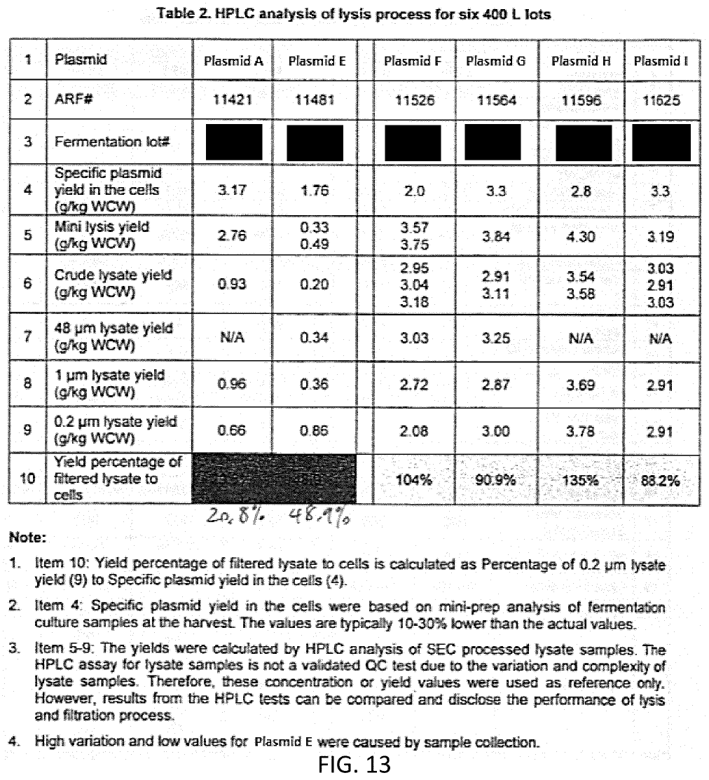

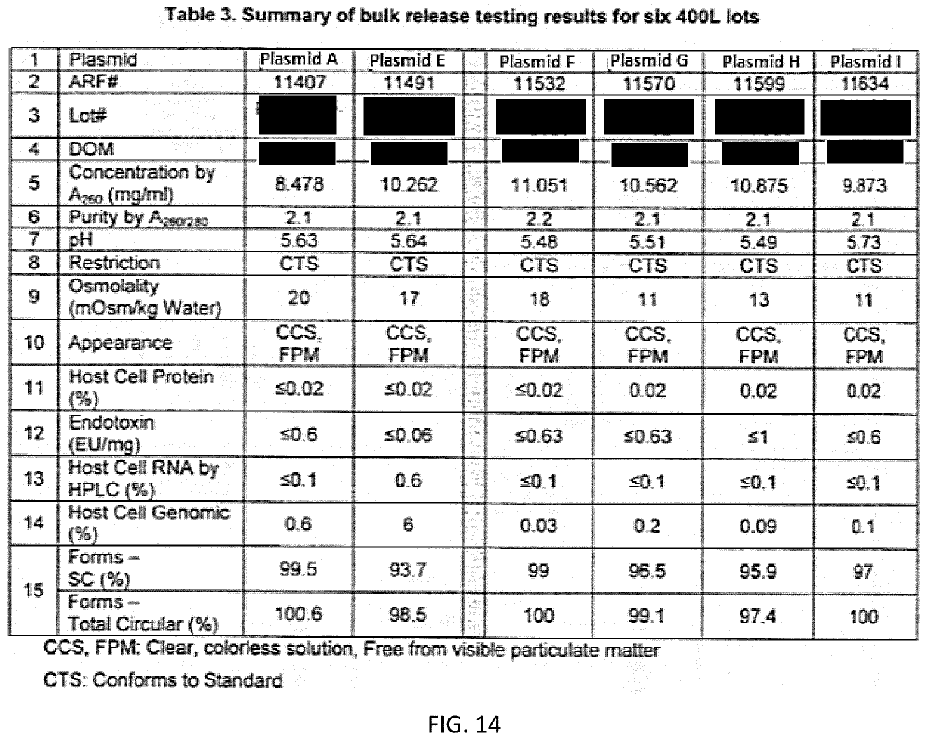

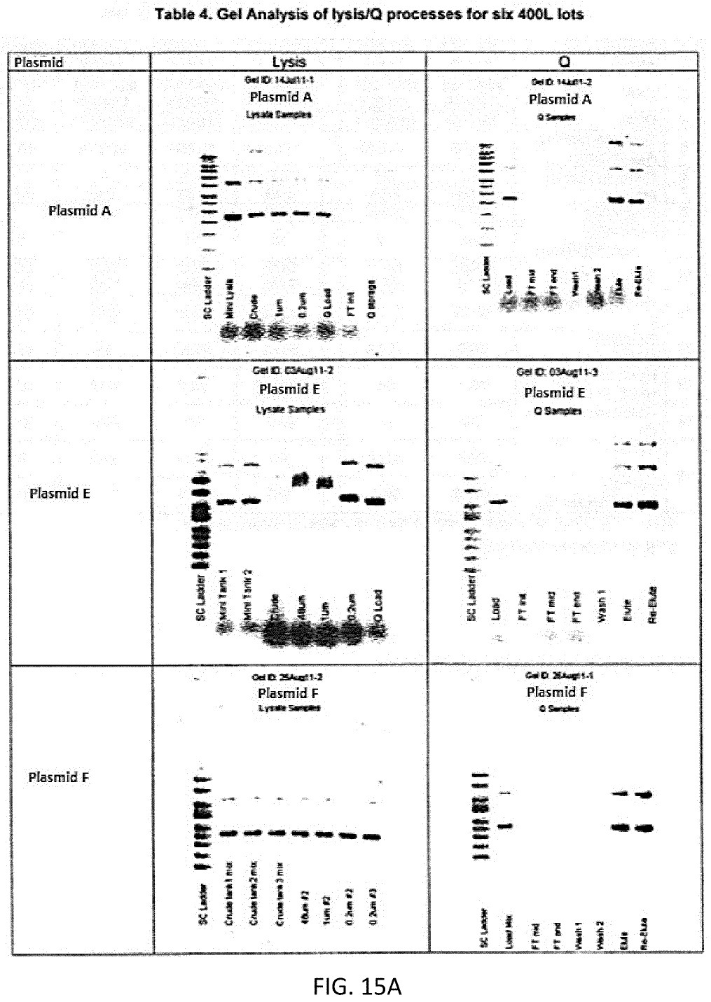

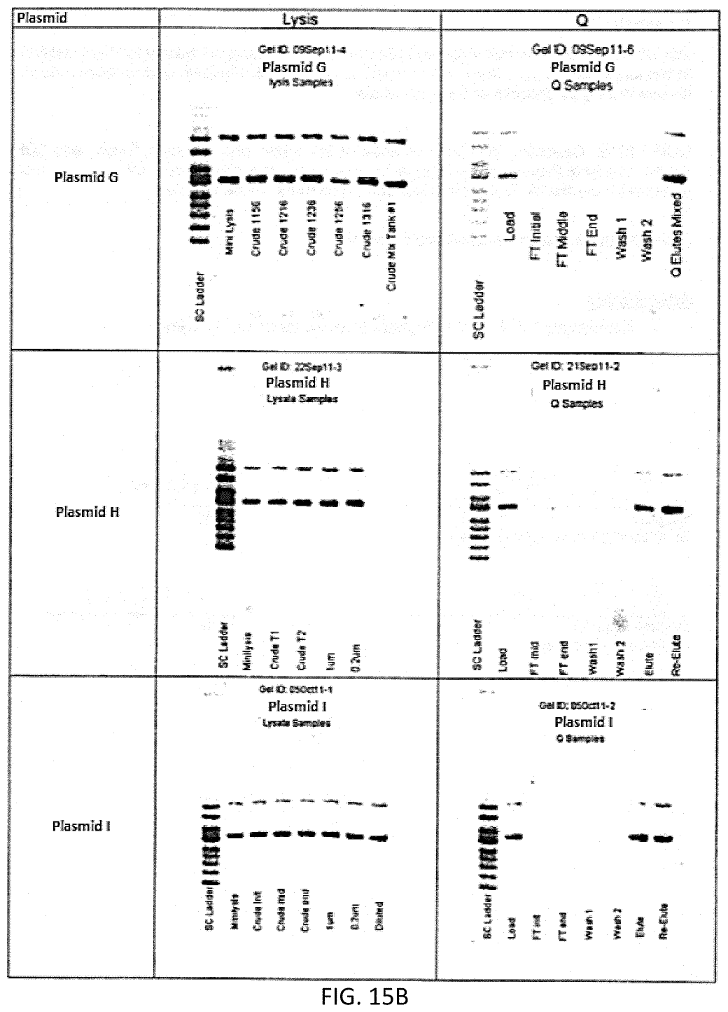

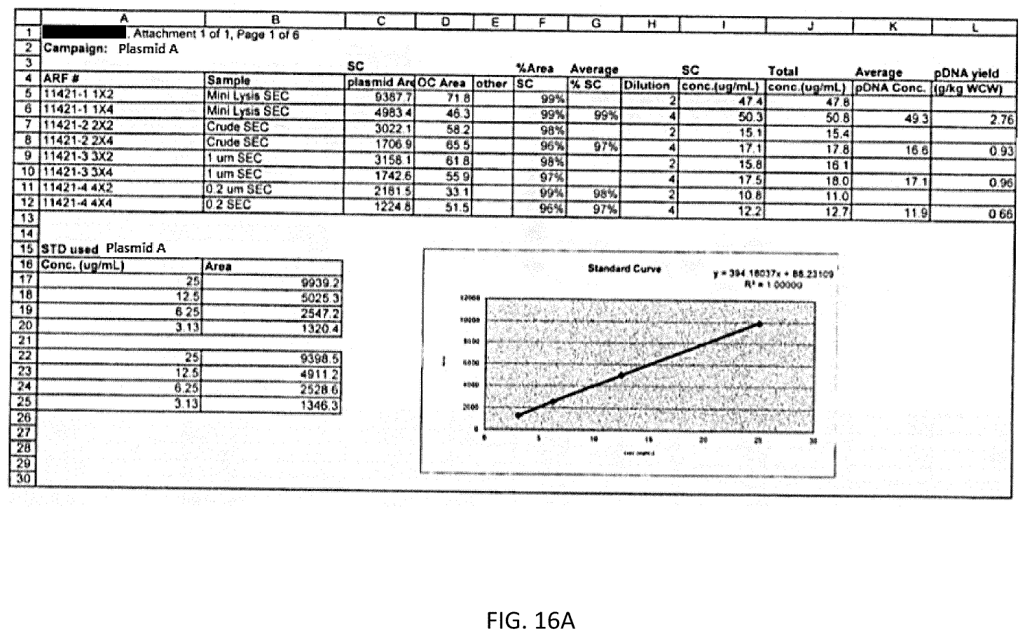

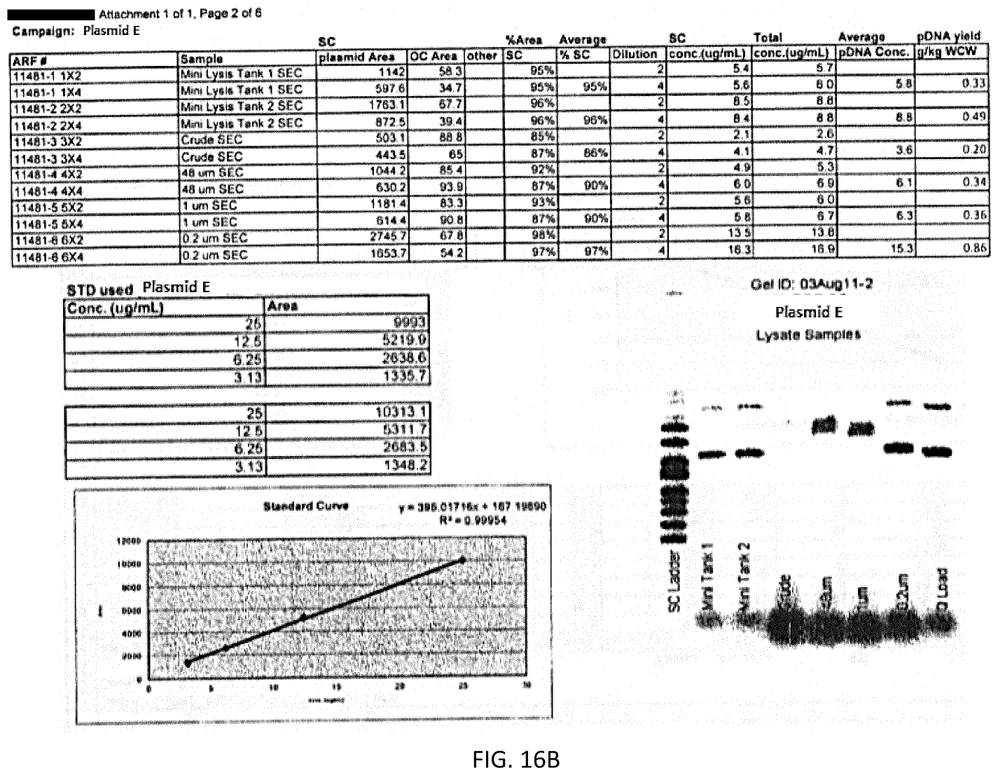

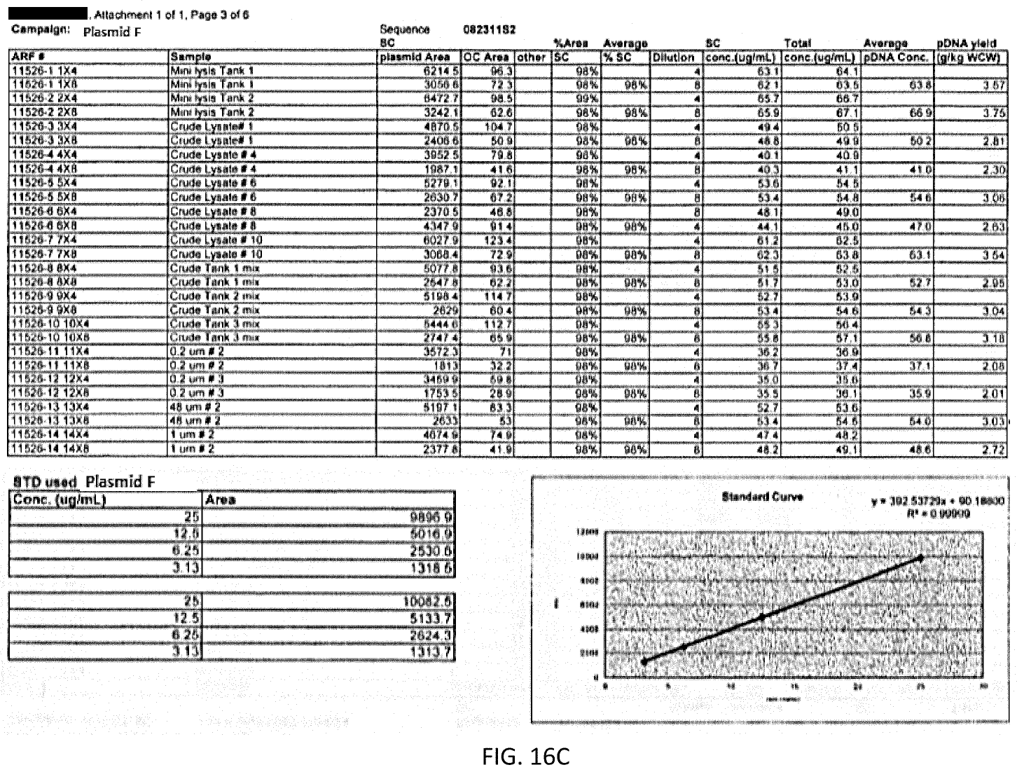

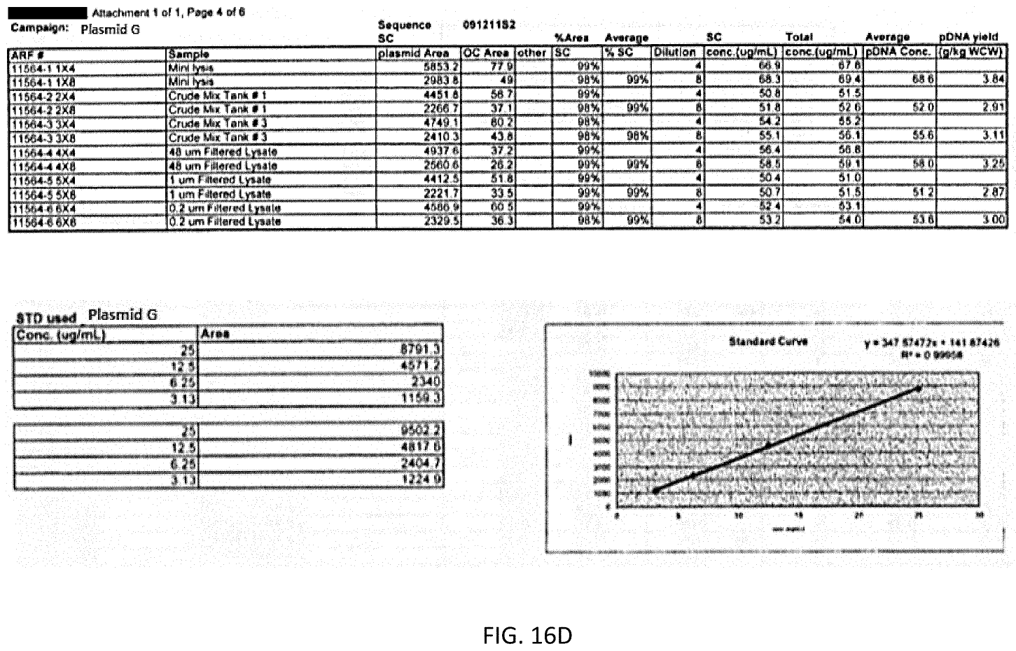

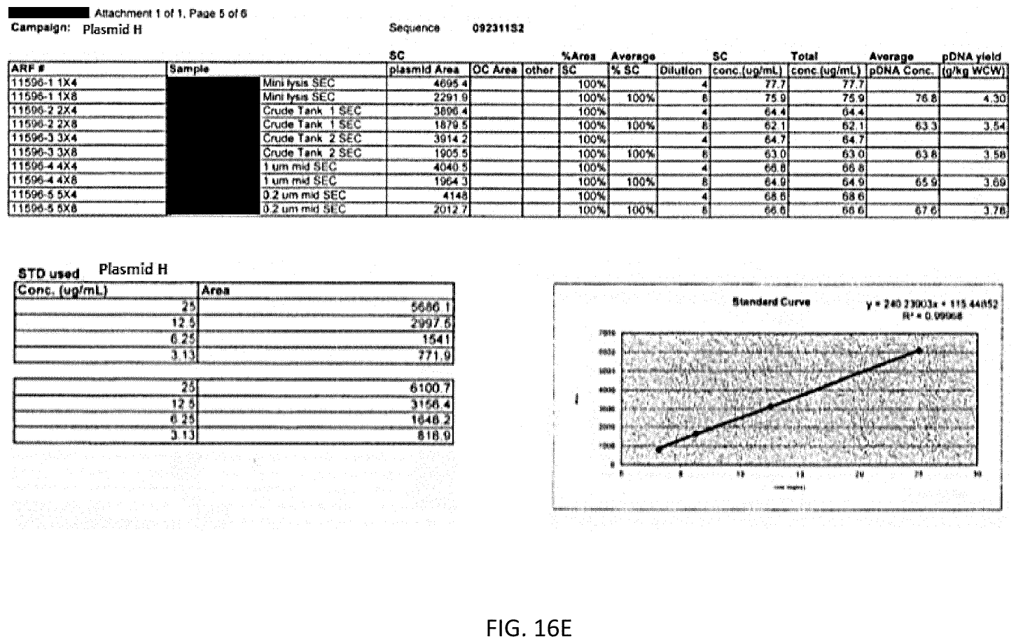

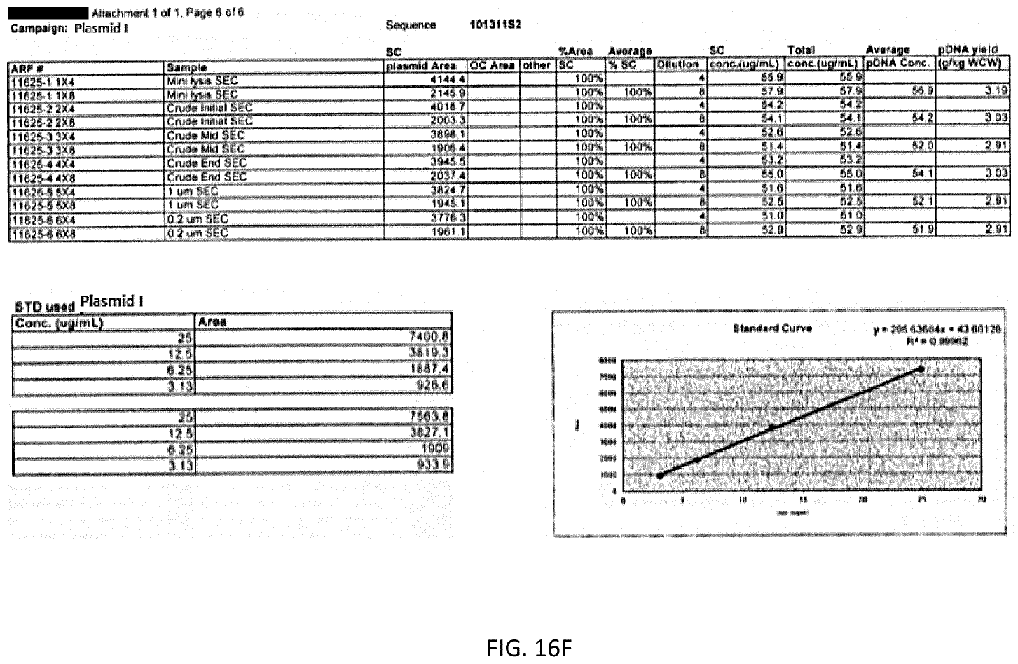

The use of the ¾″ internal diameter lysis coil improved yield and reduced variation for the lysis process compared to the use of the 1 inch internal diameter. More specifically, production runs were performed for Plasmid F, Plasmid G, Plasmid H and Plasmid I using the new coils. Purification data for six consecutive lots, 2 with 1 inch internal diameter coils and 4 with ¾ inch internal diameter coils are summarized in FIG. 12. HPLC analysis of the plasmid concentration in the lysate samples are summarized in FIG. 13. Summary of bulk release testing results for the six lots is given in FIG. 14. Gel analysis of the lysis and Q process for six lots are shown in FIG. 15A and FIG. 15B. HPLC analysis of the lysate samples for six lots are shown in FIG. 16A through FIG. 16F.

Plasmid A had a low yield of 7.4% for the overall purification process (FIG. 12, row 24). The product yield at the Q purification step was much lower than the initial estimation (FIG. 12, row 8 and 9). The root cause was identified in the lysis. Plasmid yield in the filtered lysate was only 20.8% (FIG. 13, row 10) compared to initial yield estimation by mini-prep (FIG. 13, row 4). Such plasmid loss was unusual and mostly caused by inconsistent holding time from 1 inch ID lysis coil. Gel analysis confirmed decreased plasmid concentration and high genomic background in the Q eluate (FIG. 15A and FIG. 15B). The high gDNA impurity was able to be reduced by the butyl load condition of 1:4 v/v 3M ammonium sulfate, but loss of plasmid in the lysis step could not be recovered later.

Plasmid E had an overall purification yield of lower than 15% (FIG. 12, row 24) and Q yield lower than the initial estimation (FIG. 12, row 8 and 9). Additional OOS associated with Plasmid E was the high gDNA in the bulk product (FIG. 14, row 14). Use of 1 inch ID lysis coil contributed to the decreased plasmid production in the filtered lysate (48.9%), and insufficient denaturing of gDNA. High gDNA in the lysate and Q eluate (FIG. 15A and FIG. 15B) could not be eliminated by the butyl load condition of 1:5 v/v 3M ammonium sulfate. Therefore, final product had 6% gDNA, which is 10-100 fold higher than a typical process may achieve.

Plasmid F was the first cGMP lot that implemented the ¾″ ID lysis coil. The actual yield at Q step was 61.7% (FIG. 12, row 9), which was similar to the estimation (FIG. 12, row 8). A typical overall purification yield is ˜30%. The overall yield was 21.5%, but mostly affected by product loss in the butyl step. Plasmid yield in the filtered lysate was 104% (FIG. 13, row 10) compared to initial yield estimation by mini-prep (FIG. 13, row 4). Gel analysis confirmed consistent plasmid concentration in all lysate samples, and low genomic background in the Q eluate (FIG. 15A and FIG. 15B). Final bulk release testing results demonstrated low impurity profiles, particularly gDNA: 0.03% (FIG. 14, row 14). The use of the ¾″ lysis coil prevented the potential problems of low lysis yield and high gDNA for Plasmid F. High yield (up to the Q step) and high quality product were achieved.

Plasmid G run also implemented the ¾″ lysis coil. The actual yield at Q step was 58.5% (FIG. 12, row 9), which was higher than the estimation (FIG. 12, row 8). The overall purification yield was 44.0%, higher than the previous 3 purification runs. Plasmid yield in the filtered lysate was 90.9% (FIG. 13, row 10), which was consistent with initial yield estimation (FIG. 13, row 4). Gel analysis also demonstrated consistent plasmid concentration in all lysate samples, and low genomic background in the Q eluate (FIG. 15A and FIG. 15B). Butyl load condition of 1:5 v/v 3M ammonium sulfate was used, which had little effect on gDNA reduction. However, final bulk gDNA was 0.2% (FIG. 14, row 14). Again, use of the ¾″ ID lysis coil achieved high yield (Q step and overall) and high quality product for plasmid Plasmid G.

Similar results were achieved for Plasmid H compared to Plasmid G. High yields (Q step and overall) and high quality product were demonstrated by using the ¾″ ID lysis coil.

Plasmid I had a slightly lower yield at the Q step (FIG. 12, row 9), but it was assumed to be associated with the specific Q capsule. Q step and overall and high quality. Plasmid yield in the filtered lysate was 88.2% (FIG. 13, row 10) compared to the initial yield estimation (FIG. 13, row 4). Miniprep variation and concentration drop by filtration were expected, and greater than or equal to 80% of yield percentage is normal. Butyl load condition of 1:5 v/v 3M ammonium sulfate was also used, and final bulk gDNA was 0.2% (FIG. 14). Therefore, the use of ¾″ ID lysis coil also achieved good yield and high quality product for Plasmid I.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A lysis coil apparatus capable of fluidly receiving a solution of cell suspension and a lysis solution, and fluidly transferring said solutions as a solution mixture thereby lysing and releasing contents of cells in the cell suspension, comprising: a cylindrical lysis coil holder having a height, and a flexible lysis coil having a first end, a second end, and a length in-between, said flexible lysis coil configured to receive a solution of cell suspension and a lysis solution from the first end and transferring said mixture solution out of the lysis coil from the second end; wherein said lysis coil holder is capable of receiving and securing a flexible lysis coil onto outer surface of said cylindrical lysis coil holder.

2. The lysis coil apparatus of claim 1, wherein the lysis coil holder has a surface embedded with a uniform helical groove having a length traversing the height of the lysis coil holder, wherein said flexible lysis coil has an interior diameter of a size that enable the lysis coil to be received by the groove of the lysis coil holder and traverses the length of the groove of said lysis coil holder.

3. The lysis coil apparatus of claim 2, wherein the interior diameter of said lysis coil is less than 1 inch.

4. The lysis coil apparatus of claim 2, wherein the lysis coil is configured to allow the solution mixture to flow at a linear flow rate resulting in retention time in the lysis coil between about 4 to about 6 minutes.

5. The lysis coil apparatus of claim 2, wherein the lysis coil is configured to allow the solution mixture to flow at a linear flow rate of between 8 m/min to about 12 m/min.

6. The lysis coil apparatus of claim 2, wherein the length of the lysis coil is greater than 100 feet long.

7. The lysis coil apparatus of claim 2, wherein the lysis coil is disposable after a single use.

8. The lysis coil apparatus of claim 2, wherein the lysis coil holder has a radial diameter of about 24 inches and a height between about 3 feet to about 6 feet.

9. The lysis coil apparatus of claim 2, wherein the groove of the lysis coil holder traverses the circumference of the lysis coil holder at a pitch from about 2.15 degree to about 3.43 degree.

10. The lysis coil apparatus of claim 9, wherein the lysis coil holder has wheel supports by which the lysis coil apparatus can be readily transported.

11. The lysis coil apparatus of claim 2, wherein the interior diameter of said lysis coil is ⅜ inch, and the lysis coil is configured to allow the solution mixture to flow at a linear flow rate of about 9.75 m/min.

12. The lysis coil apparatus of claim 4, wherein the retention time is about 5 minutes, and the length of the lysis coil is about 150 feet long.

13. The lysis coil apparatus of claim 5, wherein the linear flow rate is about 9.75 m/min and the length of the lysis coil is about 150 feet long.

14. The lysis coil apparatus of claim 6, wherein the length is about 150 feet long and the lysis coil is configured to allow the solution mixture to flow at a linear flow rate of about 9.75 m/min.

15. The lysis coil apparatus of claim 7, wherein the lysis coil interior diameter is ⅜ inch and the lysis coil length is about 150 feet long.

16. The lysis coil apparatus of claim 2, wherein the interior diameter of said lysis coil is ¾ inch, and the lysis coil is configured to allow the solution mixture to flow at a linear flow rate of about 9.75 m/min.

17. The lysis coil apparatus of claim 16, wherein the length of the lysis coil is about 160 feet long.

18. A method of lysing cells containing a desired polynucleotide using a lysis coil apparatus capable of fluidly receiving a solution of cell suspension and a lysis solution, and fluidly transferring said solutions as a solution mixture into contact with a neutralizing solution thereby lysing and releasing contents of cells in the cell suspension, comprising: a cylindrical lysis coil holder having a height, and a flexible disposable lysis coil having a first end, a second end, and a length in-between, said flexible lysis coil configured to receive a solution of cell suspension and a lysis solution from the first end and transferring said mixture solution out of the lysis coil from the second end; wherein said lysis coil holder has a surface embedded with a uniform helical groove having a length traversing the height of the lysis coil holder with a consistent pitch; and wherein said flexible lysis coil has an interior diameter of a size that enables the lysis coil to be received by the groove of the lysis coil holder and traverses the length of the groove of said lysis coil holder, comprising the steps:
securing a disposable lysis coil into the groove of the lysis coil holder;
transferring the solution of cell suspension into the first end of the lysis coil;
transferring the lysis solution into the first end of the lysis coil to enable the solution of the cell suspension to mix with the lysis solution; and
fluidly transferring the solution mixture to a compartment along with neutralizing solution to end the lysing process.

19. The method of claim 18, wherein the transferring steps occur at a linear flow rate of from about 8 m/min to about 12 m/min.

20. The method of claim 18, wherein the mixture solution traverses the length of the lysis coil in about between 4 minutes to 6 minutes.

21. The method of claim 20, wherein the transferring steps occur at a linear flow rate of about 9.75 m/min.
